(19) United States
(12) Patent Application Publication
(10) Pub. No.: US 2017/0056644 A1
(43) Pub. Date: Mar. 2, 2017

(54) TEXTILE-BASED PRODUCT

(71) Applicants: Tony CHAHINE, Toronto (CA); Vikram SHARMA, Toronto (CA); Moshe ROCK, Toronto (CA); Gabriel STEFAN, Toronto (CA); Eric Richard ROBERT, Toronto (CA)

(72) Inventors: Tony CHAHINE, Toronto (CA); Vikram SHARMA, Toronto (CA); Moshe ROCK, Toronto (CA); Gabriel STEFAN, Toronto (CA); Eric Richard ROBERT, Toronto (CA)

(21) Appl. No.: 15/230,137

(22) Filed: Aug. 5, 2016

Related U.S. Application Data
(60) Provisional application No. 62/201,318, filed on Aug. 5, 2015.

(51) Int. Cl. A61N 1/04; A61F 7/02; A41D 27/10; A41D 1/00; A41B 1/08; A41D 1/06; A61F 7/00; A61N 1/36

(52) U.S. Cl. CPC A61N 1/0484; A61F 7/007; A61F 7/02; A61N 1/0456; A61N 1/0452; A61N 1/36014; A61N 1/36021; A61N 1/36003; A41D 1/002; A41B 1/08; A41D 1/06; A41D 27/10; A61F 2007/0071; A61F 2007/0234; A61F 2007/0236; A41D 2500/10

(57) ABSTRACT

A textile product comprising a non-conductive section comprising a network of non-conductive fibres and an electric pathway comprising a network of conductive fibres, the electric pathway for conducting or transmitting an electrical signal when connected to a power source, is provided herein. The electric pathway and the non-conductive section are integrated into a common layer of the textile.

### CROSS-REFERENCE TO RELATED APPLICATIONS

This application claims the benefit of, and priority to, U.S. Provisional Patent Application No. 62/201,318, filed Aug. 5, 2015, the entire contents of which is hereby incorporated herein by express reference thereto.

### TECHNICAL FIELD

This document relates to the technical field of a textile product and methods for manufacturing therefor.

### BACKGROUND

A medical treatment device includes (for example) an electronic stimulation device configured to provide effective treatments for various medical therapies and/or medical treatments (for parts of the human or animal body, such as the muscles and/or the nerves and/or wounds and/or blood circulation). Electronic stimulation can also be called electrical stimulation, electrical muscle stimulation, neuromuscular electrical stimulation (NMES), electromyostimulation, neuro-stimulation, transcutaneous muscle therapy, transcutaneous muscle therapy, subcutaneous electrical stimulation, transcutaneous electrical muscle stimulation, and any equivalent thereof. Medical studies and reports have demonstrated the effectiveness and the efficacy for the usage of the electronic stimulation device. The purpose of this is also important for; wound healing because it generates a subtle electric field, which provides continuous electric stimulation that has anti-bacterial effects as well as promotes healing of chronic wounds and ulcers.

Electronic stimulation (electrical muscle stimulation is the elicitation of muscle contraction using electric impulses. Electronic stimulation has received increasing attention in the last few years because of its potential to serve as (A) a strength training tool for healthy subjects and athletes, (B) a rehabilitation and preventive tool for partially or totally immobilized patients, (C) a testing tool for evaluating the neural and/or muscular function in vivo, and/or (D) a post-exercise recovery tool for athletes. Electronic stimulation impulses are generated by a device (a controller), and are delivered through electrodes placed on (coupled to) the skin (of the user receiving treatment) in direct proximity to the muscles and/or nerves to be stimulated. The electronic stimulation impulses mimic the action potential coming from the central nervous system thereby causing the muscles to contract, etc. The electrodes are generally pads that adhere to the skin. The use of electronic stimulation has been cited by sports scientists as a complementary technique for sports training and published research is available on the results obtained. Electronic stimulation devices can be regulated by various government regulating agencies. Luigi Galvani (circa 1) provided the first scientific evidence that electrical current can activate the muscle of a person. Since then, researchers have studied and documented the exact electrical properties that generate muscle movement. It was discovered that the body functions induced by electrical stimulation caused long-term changes in the muscles. Sport scientists have applied electronic stimulation in the training of elite athletes. Electrical stimulation causes adaptation of cells of muscles, blood vessels and nerves.

It is advantageous to apply electronic stimulation to an afflicted area (such as, to a portion of a muscle of the user), a therapy area and/or a portion of the nervous system of the user (and any equivalent thereof). Electronic stimulation can be performed or applied by (A) placing a pair of electrodes on a specific body part or area (of the user), and (B) conducting electrical simulation pulses in the surrounding tissue (this is done in such a way that pain associated with the body part can be managed and/or therapy can be provided to the body part (therapeutic benefit, etc.).

Existing textile products with conductive elements for heating, as illustrated in US Patent App. No. U.S. 20080245786, incorporate individual conductive elements at symmetrical and asymmetrical pattern for uniform heating.

Existing products for patterned and controlled heating are external patches that are generated via cutting (e.g. stamping out) of patterns on a conductive fabric. This limitation requires multiple additional steps to generate a patterned heating element. Furthermore, this creates an uncomfortable package as the heating elements are an additional layer that is applied to the existing textile garment or product.

Existing EMS (Electrical Muscle Stimulation)/TENS(Transcutaneous electrical nerve stimulation)/ENS(Early Neurological Stimulation) products are rubber patches that are first attached to the skin then connected to electrical power to transmit a signal or stimulation to the skin. External wires are attached to the conductive patches and power source. The customer has to peel off the patches after the treatment which can be uncomfortable as hair is ensnared with the patches). Such systems are require a change in patches after few uses and as such are inconvenient as they are “add-ons” to an existing garment.

The electrode assembly includes an electrode coupled to (supported by) a pad. The electrode assembly is configured to operatively contact the surface (the skin) of the user (the patient). In such medical treatment devices, contact with the electrode assembly can cause unwanted irritation to the skin of the patient. The electrode assembly can be used on a user (such as, a human or an animal).

While the known electrode assembly work well enough, the known electrode assembly cannot be suitable for day-to-day use and/or for comfortable to use.

For instance, some known electrode assemblies cannot be washed and reused (for hygienic purposes, etc.).

Some known electrical stimulation devices include a hydro-gel electrode (also called, a sticky sensor) that can cause some degree of discomfort, pain and/or skin irritation to the patient (that is, the user receiving therapy), especially for the case where the hydro-gel electrode is used over a prolonged period (due to the type of glue deployed in the electrode).

Furthermore, the known electrode assembly can be used in conjunction with known garments having an electrically-conductive network. The electrically-conductive network can include external electrical connection junctions that are not desirable for electrical transmission and/or connection integrity. The conductive network can be called an electrical conductive circuit or built-in electrical wiring, etc.

Attaching the known electrode assembly to existing garments can be accomplished by using manufacturing techniques (such as, sewing, embroidery, etc.), and these arrangements cannot provide a configuration for effective transfer of electrical stimulation to the skin of the user. The junctions for attaching electrical leads from the electrode assembly to the electrical circuit of the garment (to be worn by the user) can have limitations for applicability and integrity.

In addition, there is a disadvantage for connecting electrode assembly and/or a sensor (such as, a heart pulse rate detection sensor) to the electrical circuit of the garment (in terms of a less-than-effective product life span).

### SUMMARY

It will be appreciated that there exists a need to mitigate (at least in part) at least one problem associated with the existing textile-based products.

In accordance with an embodiment, the existing textile-based products can include (and are not limited) to garments configured to be worn by users, and/or with existing medical treatment devices (also called the existing technology).

In accordance with an embodiment, the textile product can be tailored and/or designed such that the product can be used by a user (such as, a person, a pet, an animal) for the defined benefit that can be provided by usage of the integrated functionality of medical treatment devices in (with) the textile structure.

Medical treatment devices (such as, electronic stimulation devices) are configured to provide a controlled electrical current (input sensory stimulus) through (via) an electrode assembly. The electrode assembly is placed on (positioned on and coupled to) the surface of the body (of the user). In this manner, the controlled electrical current is then activated. This is done in such way that effective therapy is provided (such as, repeated muscular contraction of a muscle positioned proximate to or underlying the electrode. Specifically, the input sensory stimulus is applied to a portion of the muscles and/or the nerves of the user.

The definition of the electrode assembly is any device (sensor, transducer, wire, etc.) that is configured to convey (transmit and/or receive) a signal between the electrical circuit (of a medical device) and the user (such as, the skin of the user).

Seamless garments with electrode-connection systems that are (directly) attached on the garment fabric surface also use a mechanical connection device and/or a chemical connection mechanism.

The electrode is kept in direct contact with the skin of the body (of the user) by the construction or configuration of the textile based product (such as, the garment, etc.).

The electrical connection between the electrode (of the sensor) and the integrated electrically-conductive network (circuit) is configured to relay electronic signals (electronic data) from the electrode (of the sensor) to a controller (computer).

In addition, a mechanical connector and/or a chemical connector typically are used to make an electrical connection between the sensor and the conductive network of the fabric.

It will be appreciated that the existing technology is associated with many technical limitations that hamper or degrade the treatment effectiveness of the known electrical stimulation products configured to provide electronic stimulation to a user. In view of the foregoing, in order to mitigate (at least in part) at least one or more problems associated with the existing technology is an aspect of a textile-based product. The textile-based product can be used by a user (such as, a human or an animal). The textile-based product includes (and is not limited to) any one of a knitted textile, a woven textile, or a cut and sewn textile, a garment, a knitted fabric, a non-knitted fabric, a material that can or cannot contact the user, a mat, a pad, a seat cover, etc.; in any combination and/or permutation thereof (any equivalent thereof). The textile-based product can include an integrated functional textile article, it will be appreciated that some embodiments described a knitted garment fabric, and it is understood that these embodiments can be extended to any textile fabric forms and/or techniques such as (weaving, knitting—warp, weft etc.), and the embodiments are not limited to a knitted garment fabric. It will be appreciated that (where indicated) the FIGS (drawings) can be directed to a knitted garment fabric; and it will be appreciated that the knitted garment fabric is an example of any form of textile fabrics forms and techniques such as (weaving, knitting—warp, weft etc.), and that any description and/or illustration to the knitted garment fabric does this limit the scope of the present invention. In accordance with an embodiment, there is provided a textile fabric garment made with any textile forming technique (and the knitted fabric garment is simply an example of such an arrangement.

In accordance with an embodiment, the textile-based product can include a user garment that is for use with an electronic stimulation device having an electronic stimulation sensor and an electronic stimulation controller, and is also for use with a user. The electronic stimulation sensor can be called a sensor, an electrode, sensor pad, etc. As such; the term garment and textile product can be used interchangeably.

The user garment includes (comprising) a synergistic combination of a knitted garment fabric (a knitted garment fabric) and a knitted electrical circuit (also called a knitted seamless electrical circuit). The user garment is not limited to a knitted garment-garment, and can be woven with a knitted portion, etc. The knitted garment fabric is configured to be (A) worn (at least in part) by the user; and (B) skin compatible with skin of the user once the user wears the knitted garment fabric.

The knitted electrical circuit is fully integrated with the knitted/woven (or otherwise integrated in a single layer) garment fabric. The knitted electrical circuit is configured to be: (A) operatively connectable to the electronic stimulation sensor and to the electronic stimulation controller in such a way that the knitted electrical circuit electrically connects the electronic stimulation sensor with the electronic stimulation controller; and (B) skin compatible with the skin of the user wearing the knitted garment fabric.

In accordance with an option of the first embodiment, the knitted garment fabric is configured to provide a controlled compression. In this manner, the knitted garment fabric is configured to provide a desired level (amount) of skin-contact force to the electronic stimulation sensor.

In accordance with an option of the first embodiment, the electronic stimulation sensor can be constructed with and/or integrated in the knitted electrical circuit.

In accordance with an option of the first embodiment, the knitted electrical circuit includes an integrated knitted heating system. The integrated knitted heating system is configured to be skin compatible with the skin of the user wearing the knitted garment fabric.

More specifically, the integrated knitted or woven heating system is configured receive (in use) an electrical current from the knitted electrical circuit. The integrated knitted heating system is also configured to provide (in use) heat (to the user wearing the knitted garment fabric) in response to receiving the electrical current. In this manner, the heat that is generated by the integrated knitted heating system can be provided to the skin of the user wearing or being in contact with the knitted garment fabric.

In accordance with a second major embodiment, the user garment is for use with a user.

In accordance with another embodiment, the user garment includes (comprising) a synergetic combination of a knitted garment fabric and a knitted electrical circuit (also called, a knitted seamless electrical circuit) and an integrated knitted heating system (also called an integrated knitted heating system).

The knitted garment fabric is configured to be (A) worn (at least in part) by the user; and (B) skin compatible with skin of the user once the user wears the knitted garment fabric and any non-garment product as well.

The knitted electrical circuit is fully integrated with the knitted garment fabric. The knitted electrical circuit is skin compatible with the skin of the user wearing the knitted garment fabric.

The integrated knitted heating system is operatively coupled to the knitted electrical circuit. The integrated knitted heating system is configured to (A) receive, in use, an electrical current from the knitted electrical circuit; (B) provide, in use, heat in response to receiving the electrical current; and (C) be skin compatible with the skin of the user wearing the knitted garment fabric.

The knitted garment fabric (of any one of the first major embodiment and the second major embodiment) is preferably configured to include a textile material that can be used in regular life activity.

The knitted or woven garment fabric can include a sleeve, a brace, a pad, a shirt, a pant, etc.

Preferably, the knitted or woven garment fabric is configured to be wore by the user out of (away from) the house or out of (away from) a medical clinic.

In accordance with an option of any one of the first embodiment and the second embodiment, the user garment further includes a power source (such as a battery) configured to be attachable to and supported by the knitted garment fabric.

In accordance with an option of any one of the first embodiment and the second embodiment, the user garment further includes an electronic stimulation controller configured to be attachable to and supported by the knitted garment fabric.

In accordance with an option of any one of the first embodiment and the second embodiment, the user garment further includes an electronic stimulation controller configured to be attachable to and supported by the knitted garment fabric (such as, a silhouette).

For the case where the user garment is used (activated) to provide heat to the user wearing the knitted garment fabric and/or for the case where the user garment is used (activated) to provide electronic stimulation to the user wearing the knitted garment fabric, the user garment can enhance the healing process of an aching muscle (of the user) as the user goes about a variety of daily activity (such as, working, resting, walking, exercising, etc.).

For the case where a further reduction in the healing time associated with the treatment of a muscle ache or joint inflammation (of the user) is required, the user garment further includes an integrated knitted heating system embedded in a textile of the knitted garment fabric (the knitted garment (not just garment—textiles in general) fabric can include a sleeve, a brace or a pad, etc. or a gauze like the one doctor uses when covering a wound or before they apply the cast on a broken bone: i.e., a wrap).

For the case where a further reduction in the healing time associated with the treatment of a muscle ache or joint inflammation (of the user) is required, the user garment further includes an integrated knitted heating system embedded in a textile of the knitted garment fabric (the knitted garment fabric can include a sleeve, a brace or a pad, etc.

It will be appreciated that the application of heat and electronic stimulation to the user wearing the knitted garment fabric can be combined together with the knitted garment fabric.

Other aspects are identified in the claims.

Other aspects and features of the non-limiting embodiments can now become apparent to those skilled in the art upon review of the following detailed description of the non-limiting embodiments with the accompanying drawings.

A first aspect provided is a textile product comprising: a non-conductive section comprising a network of non-conductive fibres; and an electric pathway for conducting or transmitting an electrical signal when connected to a power source via a first connector and a second connector, the electric pathway and the non-conductive section integrated into a common layer of the textile, the electric pathway comprising: a first conductive segment of the electric pathway for coupling with the power source via the first connector, the first conductive segment comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source, the first conductive segment having a first electrical resistance; and a second conductive segment of the electric pathway for coupling with the power supply via the second connector, the second conductive segment comprising a second network of conductive fibres having a plurality of third conductive fibres, at least one third conductive fibre coupled to the second connector along the electric pathway, and a plurality of fourth conductive fibres interlaced with the third conductive fibres extending lateral to the pathway, the second conductive segment having a second electrical resistance differing from the first electrical resistance.

A second aspect provided is a textile product of claim wherein the first conductive segment and the second conductive segment are arranged in series such that the electric signal is transmitted from the first network of conductive fibres to the second network of conductive fibres.

A third aspect provided is the second conductive segment being attached directly to the second connector via the at least one third conductive fibre or the second conductive segment being attached indirectly to the second connector via a third conductive segment coupled to the second conductive segment, the third conductive segment directly attached to the second connector.

A fourth aspect provided is a textile product of claim wherein the first conductive segment is attached indirectly to the first connector via a third conductive segment coupled to the first conductive segment, the third conductive segment directly attached to the first connector.

A fifth aspect provided is a textile product of claim further comprising a second electric pathway for conducting or transmitting a second electrical signal when connected to the power source, the second electric pathway and the non-conductive section integrated into the common layer of the textile; the second electric pathway comprising: a first stimulating conductive segment for coupling with the power supply via a first stimulating connector, the first stimulating conductive segment comprising a first stimulating network of conductive fibres having a plurality of first stimulating conductive fibres, at least one first stimulating conductive fibre coupled to the first stimulating connector along the second electric pathway, and a plurality of second stimulating conductive fibres interlaced with the first stimulating conductive fibres extending lateral to the second electric pathway to transmit the second electric signal from the power source; and a second stimulating conductive segment as an electrode and for coupling with the power supply via a second stimulating connector, the second stimulating conductive segment comprising a second stimulating network of conductive fibres having a plurality of third stimulating conductive fibres, at least one third stimulating conductive fibre coupled to the second stimulating connector along the second electric pathway, and a plurality of fourth stimulating conductive fibres interlaced with the third stimulating conductive fibres extending lateral to the second electric pathway; wherein the electrode is configured to deliver the second electric signal to an adjacent underlying body portion of a wearer of the textile.

A sixth aspect provided is a textile product comprising: a first conductive segment for coupling with a power supply via a first connector and a second connector attached to an electric pathway, the first conductive segment of the electric pathway comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source, the first conductive segment having a first electrical resistance; and a second conductive segment of the electric pathway for coupling with the power supply via the second connector, the second conductive segment having a second network of conductive fibres having a plurality of third conductive fibres, at one third conductive fibre coupled to the second connector along the electric pathway, and a plurality of fourth conductive fibres interlaced with the third conductive fibres extending lateral to the pathway, the second conductive segment having a second electrical resistance differing from the first electrical resistance; the first and second conductive segments of the electric pathway integrated into a common layer of the textile.

A sixth aspect provided is a textile product comprising: a non-conductive section comprising a network of non-conductive fibres; and an electric pathway for conducting or transmitting an electrical signal when coupled to a power source via a first connector and a second connector attached to the electric pathway, the electric pathway and the non-conductive section integrated into a common layer of the textile; the electric pathway comprising: a first conductive segment of the electric pathway for coupling with the power supply via the first connector, the first conductive segment comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source; and

a second conductive segment configured as an electrode of the electric pathway and for coupling via the second connector, the second conductive segment comprising a second network of conductive fibres having a plurality of third conductive fibres, at least one third conductive fibre coupled the second connector along the electric pathway, and a plurality of fourth conductive fibres interlaced with the third conductive fibres extending lateral to the pathway; wherein the electrode is configured to deliver the electric signal to an adjacent underlying body portion of a wearer of the textile.

A seventh aspect provided is a mixed layer textile product.

An eighth aspect provided is a textile product having only one conductive segment interlaced in a fabric layer of the textile product coupled to a first connector and a second connector attached to a power source.

### BRIEF DESCRIPTION OF THE DRAWINGS

The non-limiting embodiments can be more fully appreciated by reference to the following detailed description of the non-limiting embodiments when taken in conjunction with the accompanying drawings, in which:

FIG. 1A and FIG. 1B depict schematic views of embodiments of an apparatus having a textile-based product (such as, a knitted garment fabric);

FIG. 2 depicts a schematic view of an embodiment of an apparatus having a textile-based product (such as, a knitted garment fabric);

FIG. 3 depicts a schematic view of an embodiment of an apparatus having a textile-based product (such as, a knitted garment fabric);

FIG. 4 depicts a schematic view of an embodiment of an apparatus having a textile-based product (such as, a knitted garment fabric);

FIG. 5 depicts a schematic view of an embodiment of an apparatus having a textile-based product (such as, a knitted garment fabric);

FIG. 6A and FIG. 6B depict schematic views of embodiments of an apparatus having a textile-based product (such as, a knitted garment fabric);

FIG. 7A and FIG. 7B depict schematic views of embodiments of an apparatus having a textile-based product (such as, a knitted garment fabric);

FIG. 8A and FIG. 8B depict schematic views of embodiments of an apparatus having a textile-based product (such as, a knitted garment fabric);

FIG. 9 depicts a schematic view of an embodiment of an apparatus having a textile-based product (such as, a knitted garment fabric);

FIG. 10 depicts a schematic view of an embodiment of an apparatus having a textile-based product (such as, a knitted garment fabric);

FIG. 11 depicts a schematic view of an embodiment of an apparatus having a textile-based product (such as, a knitted garment fabric);

FIG. 12 depicts a schematic view of an embodiment of an apparatus having a knitted garment fabric;

FIG. 13 depicts a schematic view of an embodiment of an apparatus having a knitted garment fabric;

FIG. 14 depicts a schematic view of an embodiment of an apparatus having a knitted garment fabric;

FIG. 15 depicts a schematic view of an embodiment of an apparatus having a knitted garment fabric;

FIG. 16 and FIG. 17 depict schematic views of embodiments of an apparatus having a knitted garment fabric;

FIG. 18 depicts a schematic view of an embodiment of an apparatus having a knitted garment fabric;

FIG. 19 and FIG. 20 depict schematic views of embodiments of an apparatus having a knitted garment fabric;

FIG. 21A and FIG. 21B depict schematic views of embodiments of an apparatus having a knitted garment fabric;

FIG. 22 depicts a schematic view of an embodiment of an apparatus having a textile-based product (such as, a knitted garment fabric);

FIG. 23A depicts a schematic view of an embodiment of an apparatus having a textile-based product (such as, a knitted garment fabric);

FIGS. 23B to 23E depict schematic views of an embodiment of an apparatus having a knitted garment fabric;

FIG. 24 depicts a schematic view of an embodiment of an apparatus having a knitted garment fabric;

FIG. 25 depicts a schematic view of an embodiment of an apparatus having a knitted garment fabric;

FIG. 26 depicts a schematic view of an embodiment of an apparatus having a knitted garment fabric;

FIG. 27, FIG. 28 and FIG. 29 depict schematic views of embodiments of an apparatus having a knitted garment fabric;

FIG. 30 and FIG. 31 depict schematic views of embodiments of an apparatus having a knitted garment fabric;

FIG. 32, FIG. 33 and FIG. 34 depict schematic views of embodiments of an apparatus having a knitted garment fabric;

FIG. 35A and FIG. 35B depict schematic views of an embodiment of an apparatus having a knitted garment fabric;

FIG. 36 depicts a schematic view of an embodiment of an apparatus having a knitted garment fabric;

FIG. 37 depicts a schematic view of an embodiment of an apparatus having a knitted garment fabric; and

FIG. 38 depicts a schematic view of an embodiment of an apparatus having a knitted garment fabric.

The drawings are not necessarily to scale and can be illustrated by phantom lines, diagrammatic representations and fragmentary views. In certain instances, details unnecessary for an understanding of the embodiments (and/or details that render other details difficult to perceive) can have been omitted.

Corresponding reference characters indicate corresponding components throughout the several figures of the drawings. Elements in the several figures are illustrated for simplicity and clarity and have not been drawn to scale. The dimensions of some of the elements in the figures can be emphasized relative to other elements for facilitating an understanding of the various disclosed embodiments. In addition, common, but well-understood, elements that are useful or necessary in commercially feasible embodiments are often not depicted to provide a less obstructed view of the embodiments of the present disclosure.

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the invention is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described.

The benefit of an integrated functional textile article (also referred to as product) where controlled electrical pulses, current or stimulation can be imparted or transmitted to a desired location on body (of the user) and/or the surface of the user can extend to alleviating various atrophies (muscular, neural, gland, etc.) and can be effective for combating parasites as well.

A textile fabric article can be generated with known fabric forming techniques, such as but not limited to weaving, knitting, seamless knitting, non-knitting, non-weaving, etc., and any equivalent thereof.

Electronic stimulation can help to relieve pain (experienced by the user) by modulating nerve impulses (to be received by the brain of the user) that indicate pain and require relief is required. Electrical stimulation applied through electrodes can be used for therapeutic exercises for paralyzed limbs and/or for generating (improving) limb function. Electronic stimulation can be performed by using (applying) electrodes that are attached to (coupled to) the skin (of the user). The electrodes can be made of silica gels that are adhered to the human skin. The electrodes can be made of silicone gels that are adhered to the human skin. Electronic simulation devices can be used for wound healing as well. Embodiment of the textile products described herein can be tailored for specific heating in specific regions of a conductive pathway integrated into the textile product fabric.

The (e.g. knitted) textile article (e.g. garment) fabric can be manufactured using knit and/or woven fabric technologies (such as, a circular knit machine, which can knit in one direction). The textile article fabric can be manufactured by using seamless and/or automated systems, and then cut out and incorporated into a cut-and-sew garment or other textile article/product (e.g. pad or cushion for placing next to a patient or other user. The textile fabric of the textile product can be included in any type of clothing, sports clothing, compression garments, mat pads, and any equivalent thereof, and/or any non-clothing fabric products.

A technical problem associated with the existing technology relates to the provision for providing and distributing electrical power along a garment.

For three layer garments, the inside layer touches body (of the wearer), the middle layer is and electrical insulator layer, and the outer layer supports the electrical connectors (such as, metal snaps). In accordance with an option, the middle layer includes a dielectric and/or a capacitive fabric sensor.

It is understood that these garments could be tailored for use on animals and/or humans.

In another embodiment, it is to be understood that fabrics can be created incorporating the embodiments. These fabrics, whether knit or woven, can be used in other fabric based products. For example, drapes, tents, sleeping bags, bedding, floor coverings, seat covers, etc.

In another embodiment, the inventions disclosed as being knit or woven is provided by the embroidering of the conductive yarn. It is understood that the conductive fabric patches constructed out of conductive, resistive yarn can function as a sensor, electrode in any combination and/or permutation thereof

The drawings depict variations of the surface area in 2D for changing the resistance of the conductive portions of the fabric. In another embodiment, the density of the knit or weave of the resistive yarn can be altered both in 2D or 3D. For example, by forming a raised knit, the volume of the resistive yarn can be increased to decrease the resistance. For example, the density of the knit or weave can be increased and this can decrease the resistance. This can result in a 2D surface area that appears to be the same but has a different resistance due to the density or volume of resistive yarn being knit or woven.

Electrical stimulation can offer a unique treatment option to heal complicated and recalcitrant wounds, improve flap and graft survival, and even improve surgery results. Electrical stimulation has been suggested to reduce infection, improve cellular immunity, increase perfusion, and accelerate wound healing.

Electrical stimulation is used for a variety of clinical applications, such as fracture repair, pain management, and wound healing. Several different applications of electricity have been described, including direct current (DC), alternating current (AC), high-voltage pulsed current (HVPC), and low-intensity direct current (LIDC). Physicians are probably most familiar with pulsed electromagnetic field (PEMF) for repair of fracture non-unions and transcutaneous electrical nerve stimulation (TENS) for pain control. Frequency rhythmic electrical modulation systems (FREMS) is a form of transcutaneous electrotherapy using electrical stimulation that automatically varies in terms of pulse, frequency, duration, and voltage. Even through the electrical stimulation and wound healing literature uses several different types of electrical stimulation, they all seem to have positive results. As such, it is recognized that electrical connectors can be attached to the fabric layer of the textile product containing the conductive pathway. For a complete electrical circuit including the power source, each end of the electrical pathway can be connected to a respective connector (e.g. a first connector and a second connector). Each of these first and second connectors are connected respectively to a positive and negative terminal of the power source, as is known in the art. An example of the electrical connector (e.g. first second connector) is a snap or other electrically conductive body attached on one end of the electrical pathway and also connectable to the power source.

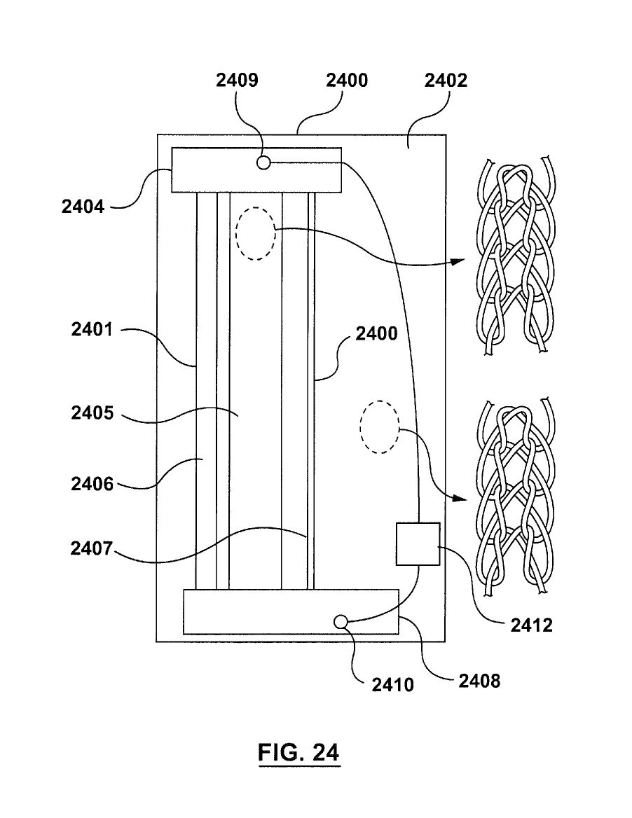

Referring to FIG. 24, two or more sections of a textile (each comprising a network or networks of fibres or yarn; e.g. an electric pathway and a non-conducting section)) can be integrated into a common layer by interlacing at least one fibre or yarn of each section with at least one fibre or yarn of an adjacent section.

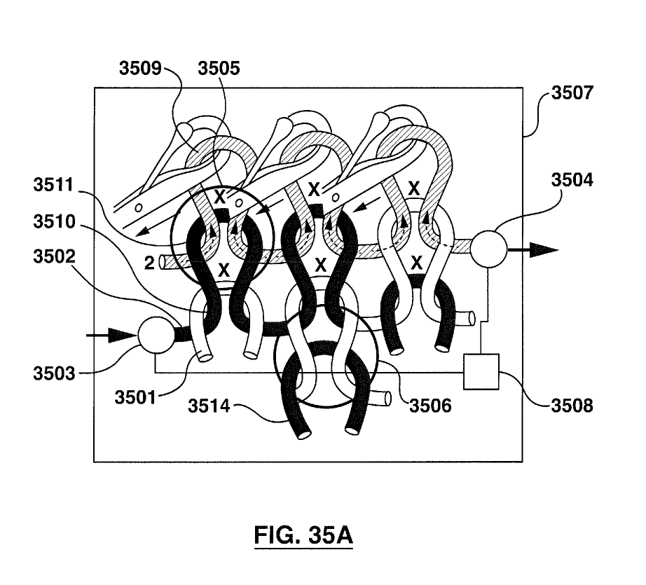

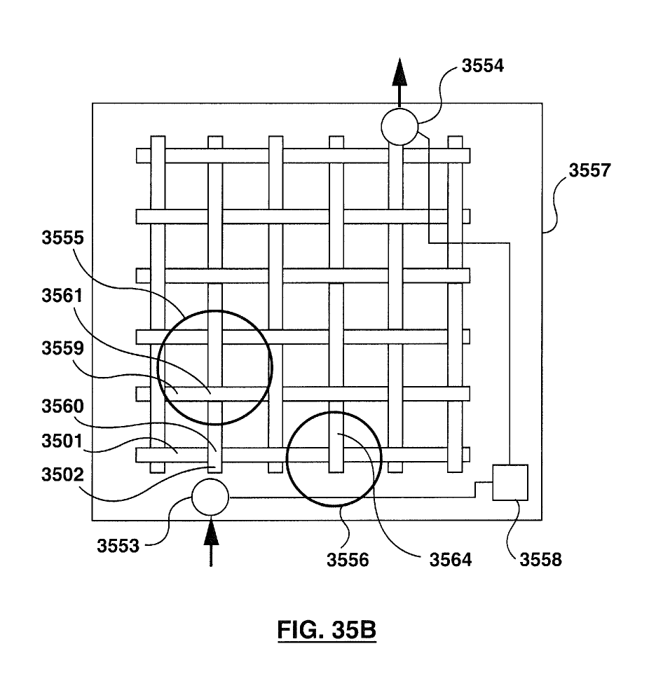

It should be noted that herein, textile refers to any material made or formed by manipulating natural or artificial fibres to interlace to create an organized network of fibres. Generally, textiles are formed using yam, where yarn refers to a long continuous length of a plurality of fibres that have been interlocked (i.e. fitting into each other, as if twined together, or twisted together). Herein, the terms fibre and yarn are used interchangeably. Fibres or yarns can be manipulated to form a textile according to any method that provides an interlaced organized network of fibres, including but not limited to weaving, knitting, sew and cut, crocheting, knotting and felting. Exemplary structures of textiles formed by knitting and weaving are provided in FIGS. 35A and 35B, respectively.

Different sections of a textile can be integrally formed into a common layer to utilize different structural properties of different types of fibres. For example, conductive fibres can be manipulated to form networks of conductive fibres and non-conductive fibres can be manipulated to form networks of non-conductive fibers. These networks of fibres can comprise different sections of a textile by integrating the networks of fibres o a common layer of the textile. Multiple layers of textile can also be stacked upon each other to provide a multi-layer textile. It is recognized that the layer of the textile is defined such that each of the fibres in the layer (for example in each section of the layer) are connected to one another in a network of fibres formed by one of the textile fabric manufacturing methods (e.g. knitting, weaving, etc.) such that each of the fibres of the network are connected to one another using the manufacturing method used to construct the textile layer. This network of fibres includes both conductive and non-conductive fibres.

It should also be noted that herein, “interlace” refers to fibres (either artificial or natural) crossing over and/or under one another in an organized fashion, typically alternately over and under one another, in a common layer. When interlaced, adjacent fibres touch each other at intersection points (e.g. points where one fibre crosses over or under another fibre). In one example, first fibres extending in a first direction can be interlaced with second fibres extending laterally or transverse to the fibres extending in the first connection. In another example, the second fibres can extend laterally at 90° from the first fibres when interlaced with the first fibres. interlaced fibres extending in a common sheet can be referred to as a network of fibres. FIGS. 35A and 35B, described below, provide exemplary embodiments of interlaced fibres. As such, it is recognized that top stitching of threads on top of the network of fibres (of the layer) is not considered as the threads being interlaced with the network of fibres. As such, top stitched threads applied to the textile fabric layer (containing the network of fibres of conductive and non-conductive threads making up the conductive pathway used for the sensors), as a separate top stitched layer additional to the textile fabric layer, is not considered to be part of the network of fibres making up the textile fabric layer.

“Integrated” refers to combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more sections comprising networks of fibres are said to be , “integrated” together into a textile (or “integrally formed”) when at least one fibre of one network is interlaced with at least one fibre of the other network such that the two networks form a common layer of the textile. Further, when integrated, two sections of a textile can also be described as being substantially inseparable from the textile. Here, “substantially inseparable” refers to the notion that separation of the sections of the textile from each other results in disassembly or destruction of the textile itself.

FIG. 24 provides a top view schematic of an exemplary electric pathway 2401 integrated with a non-conductive section 2402 within a textile 2400.

Electric pathway 2401 comprises a power source (not shown), a controller 2412, two connectors 2409, 2410 and one or more electrically conductive segments 2404, 2405, 2406, 2407 and 2408. It should be noted that electric pathway 2401 is only one example of an electric pathway and that any number of electrically conductive segments (each comprising a network of electrically conductive fibres) can be included therein.

In this embodiment, electric pathway 2401 is integrated with non-conductive section 2402 into a common layer textile 2400. “Layer” refers to a thickness of the textile. Integrating two sections (or segments of sections) into a common layer means that at least a portion of each of the two sections or segments (e.g. at least some of the fibres comprising the network of fibres of each section or segment) have a same thickness and are interlaced together to attach together at the respective portions of same thickness. As shown by the extracted portions shown to the right of FIG. 24, each of electric pathway 2401 and non-conductive section 2402 is made loops of knitted non-conductive fibres. It should be noted that electric pathway 2401 can comprise both conductive and non-conductive fibres, and non-conductive section 2402 can comprise both non-conductive fibres and conductive fibres, so long as the conductive fibres of the electric pathway 2401 are not electrically connected to the conductive fibres of non-conductive section 2402. Non-conductive section 2402 can therefore be considered as an insulator to the electric pathway 2401.

Two conductive fibres are “electrically contacting” when an electric current can be transmitted between the fibres (e.g. the adjacent fibres are touching). A conductive fibre is said to be “electrically contacting” an adjacent conductive fibre at an intersection point (see also FIGS. 35A and 35B, below).

Each of connectors 2409, 2410 is electrically connected to a power source (e.g. battery, not shown) which in turn is coupled to a controller 2412. Herein, two structures being “electrically connected” refers an attachment between the structures such that an electrical signal can be transmitted between the two structures. For example, the power source and the connectors 2409, 2410 are electrically connected to each other because there is a physical point of connection (e.g. attachment) and an electric signal can be transmitted from the battery to the connectors 2409, 2410, and vice versa.

Each of electrically conductive segments 2404, 2405, 2406, 2407 and 2408 comprise an organized network of fibres (see FIGS. 35A and 35B). Electrically conductive fibres 2404 and 2408 are shown to be electrically connected to connectors 2409 and 2410, respectively. At least a portion of the Electrically conductive segments 2404 and 2408 and connectors 2409 and 2410, respectively, can be connected by any type of conductive physical mechanism, such as a snap connector (e.g. quick snap connector), a conductive snap connector with a female portion having an insulator facing the skin of the user (as depicted as 14 in FIG. 6A, and/or as depicted as 44 in FIG. 6B), a conductive wire, a conductive adhesive material, a conductive paste, a sewing portion, a stitching, and any equivalent thereof

Electrically conductive segment 2404 is in electrical contact with electrically conductive segment 2405, where “in electrical contact” means that an electric signal can be transmitted between the segments (e.g. structures) but a physical connection does not necessarily exist. For example, electrically conductive segment 2404 can be in electrical contact with electrically conductive segment 2405 by having conductive fibres within each segment touching (e.g. crossing or overlapping). Transmission of an electric signal within an electrically conductive segment, such as electrically conductive segment 2404, is described below in reference to FIGS. 35A and 35B. It is also recognized that one or more conductive fibres can be common to both conductive segments 2404, 2405.

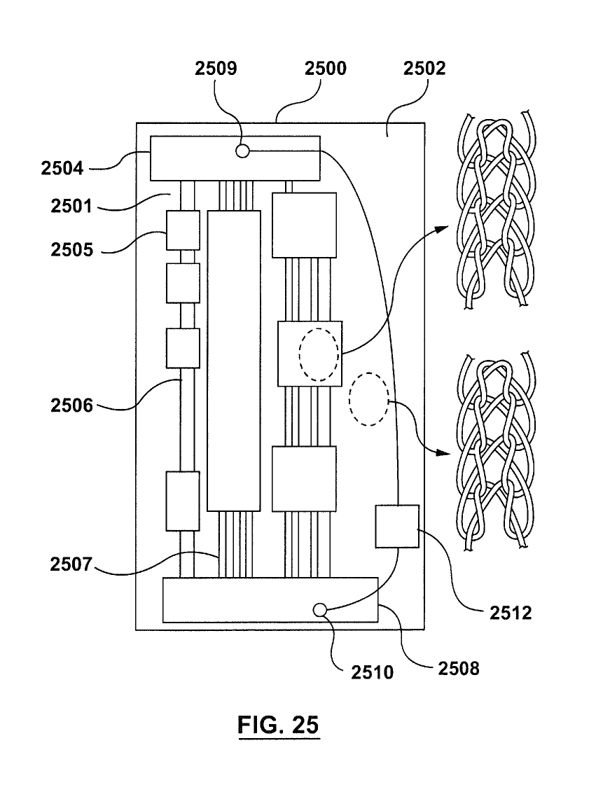

Electrically conductive segments 2404, 2405, 2406, 2407 and 2408 can configured to have varying resistances, where resistance over an electrically conductive segment (e.g. 2404, 2405, 2406, 2407 and 2408) can be controlled at least by varying the length of the segment, the width of the segment and/or the density and/or the volume of segment. The density of a segment refers to the mass of the segment per unit volume of the segment. Therefore, for example, increasing the total number of loops of conductive fibre within a unit area of an electrically conductive segment (e.g. 2404) increases the density of the electrically conductive segment As a further example, resistance increases as the width of a segment decreases. Therefore, referring to FIG. 24 for example, segment 2407 has a higher resistance (e.g. and generates more heat for a constant current and voltage) than segments 2405 and 2406 which are shown as having an increased width when compared to segment 2407. Resistance can also be controlled by varying the conductive material in the conductive fibre and the length of the conductive fibre (e.g. see FIG. 25 where segment 2506 is shown as being longer than segment 2505, therefore having a higher resistance for a same current and voltage).

In one example, FIG. 24 shows electrically conductive segments 2405, 2406 and 2407 arranged within pathway 2401 in a parallel configuration, having low, medium and high resistance, comparatively (based on their varying widths, for same currents and voltages). Electrically conductive segment 2405 is shown as the widest segment, therefore having the lowest resistance to an electric signal. Electrically conductive segment 2406 is shown as being narrower than electrically conductive segment 2405 but wider than electrically conductive segment 2407, therefore having a higher resistance than electrically conductive segment 2405 but a lower resistance than electrically conductive segment 2407. Electrically conductive segment 2407 is shown as the narrowest segment, therefore having the highest resistance of electrically conductive segments 2405, 2406 and 2407.

In operation, a power source (e.g. battery, not shown) provides an electric signal to connector 2409 upon activation from controller 2412. The power source is in electrical contact with connector 2409, so the electric signal passes from the power source through the connector 2409 into electrically conductive segment 2404. The electric signal is transferred both in the direction of electric pathway 2401 and transverse (or lateral) to electric pathway 2401. In this example, non-conductive section 2402 does not contain any electrically conductive fibres (e.g. there are no electrically conductive fibres of 2502 in electrical contact with the conducting fibres of pathway 2401), the electric signal is not transmitted beyond the fibres of electrically conductive segment 2404 into non-conductive section 2402.

In one example embodiment, knitting can be used to integrate different sections of a textile into a common layer (e.g. a conductive pathway and non-conductive sections). Knitting comprises creating multiple loops of fibre or yarn, called stitches, in a line or tube. In this manner, the fibre or yarn in knitted fabrics follows a meandering path (e.g. a course), forming loops above and below the mean path of the yarn. These meandering loops can be easily stretched in different directions. Consecutive rows of loops can be attached using interlocking loops of fibre or yarn. As each row progresses, a newly created loop of fibre or yarn is pulled through one or more loops of fibre or yarn from a prior row.

In another example embodiment, can be used to integrate different sections of a textile into a common layer (e.g. a conductive pathway and non-conductive sections). Weaving is a method of forming a textile in which two distinct sets of yarns or fibres are interlaced at right angles to form a textile.

Electrically conductive segments 2405, 2406 and 2407 are in electric contact with electrically conductive segment 2404 and arranged in series, so the electric signal passes horizontally and vertically through electrically conductive segments 2405, 2406 and 2407 to electrically conductive segment 2408.

Electrically conductive segment 2408 is electrically connected to connector 2410, which in turn is connected to the power source (e.g. battery). Upon receipt of the electric signal as segment 2408, The electric signal is transmitted from electrically conductive segment 2408 through connector 2410 and back to the power source to complete the electric circuit.

FIG. 25 provides a top view schematic of another exemplary electric pathway 2501 integrated with a non-conductive section 2502 within a textile 2500, wherein electrically conductive segments 2505, 2506, 2507 are arranged to be parallel to one another rather than in series as shown in FIG. 24.

Electric pathway 2501 comprises a power source (not shown), a controller 2512, two connectors 2509, 2510 and one or more electrically conductive segments 2504, 2505, 2506, 2507 and 2508. It should be noted that electric pathway 2501 is only one example of an electric pathway and that any number of electrically conductive fibres can be included therein.

In this embodiment, electric pathway 2501 is integrated with non-conductive section 2502 into a common layer of textile 2500. As shown by the extracted portions shown to the right of FIG. 25, each of electric pathway 2501 and non-conductive section 2502 is made loops of knitted non-conductive fibres. It should be noted that electric pathway 2501 can comprise both conductive and non-conductive fibres and non-conductive section 2502 can comprise non-conductive or conductive fibres, as long as the conductive fibres of section 2502 are not electrically connected to the conductive fibres of electric pathway 2501. Non-conductive section 2502 can therefore be considered as an insulator to the electric pathway 2501.

Each of connectors 2509, 2510 is electrically connected to a power source (e.g. battery, not shown) which in turn is coupled to controller 2512. Two structures being “electrically connected” refers being attached such that an electrical signal can be transmitted between the two structures. For example, the power source and the connectors 2509, 2510 are electrically connected to each other because there is a physical point of connection between the structures and an electric signal can be transmitted from the battery to the connectors 2509, 2510 and vice versa.

Electrically conductive segments 2504 and 2508 are also shown to be electrically connected to connectors 2509 and 2510, respectively. Electrically conductive segments 2504 and 2508 and connectors 2509 and 2510, respectively, can be connected by any type of conductive physical mechanism, such as a snap connector (e.g. a quick snap connector), a conductive snap connector with a female portion having an insulator facing the skin of the user (as depicted in FIG. 6A as item 14, and/or as depicted in FIG. 6B as item 44), a conductive wire, a conductive adhesive material, a conductive paste, a sewing portion, a stitching, and any equivalent thereof.

Electrically conductive segment 2504 is in electrical contact with electrically conductive segment 2505, where “in electrical contact” means that an electric signal can be transmitted between the segments (e.g. structures) but a physical connection does not necessarily exist. For example, electrically conductive segment 2504 can be in electrical contact with electrically conductive segment 2505 by having conductive fibres within each segment touching (e.g. crossing or overlapping). Transmission of an electric signal within an electrically conductive segment, such as electrically conductive segment 2504, is described below in reference to FIGS. 35A and 35B.

Electrically conductive segments 2504, 2505, 2506, 2507 and 2508 can be configured to have varying resistances. Resistance over an electrically conductive segment can be controlled by, for example, varying the length of the segment, varying the width of the segment and/or varying the density or volume of segment. The density of a segment refers to the mass of the segment per unit volume of the segment. Therefore, increasing the number of loops of conductive fibre within a unit area of an electrically conductive segment (e.g. 2504) will increase the density of the segment for a same current and a same voltage. For example, as shown in FIG. 25, segment 2506 is longer than segment 2505 and therefore would have a higher resistance than segment 2506 (and generate more heat) for a same voltage and a same current. Resistance can also be controlled by varying the conductive material in the conductive fibre, for example.

In one example, FIG. 25 shows electrically conductive segments 2505, 2506 and 2507 arranged within pathway 2501 in a parallel configuration, having low, medium and high resistance, comparatively (based on their length and width). Electrically conductive segment 2505 is shown as the widest segment, therefore having the lowest resistance to an electric signal. Electrically conductive segment 2506 is shown as being narrower than electrically conductive segment 2505 but wider than electrically conductive segment 2507, therefore having a higher resistance than electrically conductive segment 2505 but a lower resistance than electrically conductive segment 2507. Electrically conductive segment 2507 Is shown as the most narrow segment, therefore having the highest resistance of electrically conductive segments 2505, 2506 and 2507.

In operation, a power source (e.g. battery, not shown) provides an electric signal to connector 2509 upon activation from controller 2512. As the power source is in electrical contact with connector 2509, the electric signal passes from the power source through the connector 2509 into electrically conductive segment 2504. The electric signal is transferred in both a direction along electric pathway 2501 or a direction transverse (e.g. lateral) to electric pathway 2501. In the exemplary embodiment shown in FIG. 25, non-conductive section 2502 does not contain any electrically conductive fibres (or at least any electrically conductive fibres in section 2502 are not electrically connected to the electrically conductive fibres of pathway 2501), the electric signal is not transmitted beyond the fibres of the segments of pathway 2501 into non-conductive section 2502.

Electrically conductive segments 2505 and 2506 are in electric contact with electrically conductive segment 2504 and arranged in series, so the electric signal passes in the direction of electric pathway 2501 into through electrically conductive segments 2505 and 2506 to electrically conductive segment 2508. However, segment 2507 is parallel to segments 2505 and 2506. Therefore, the electric signal propagates out of segment 2504 and into segments 2505 and 2507 separately.

Electrically conductive segment 2508 is electrically connected to connector 2510, which in turn is connected to the power source (e.g. battery). Once received at electrically conductive segment 2508, the electric signal is therefore transmitted from electrically conductive segment 2508 through connector 2510 and back to the power source to complete the electric circuit.

FIG. 35A shows an exemplary knitted configuration of a network of electrically conductive fibres 3505 in, for example, a segment of an electric pathway (e.g. 2401). In this embodiment, an electric signal (e.g. current) is transmitted to conductive fibre 3502 from a power source (not shown) through a first connector 3505, as controlled by a controller 3508. The electric signal is transmitted along the electric pathway along conductive fibre 3502 past non-conductive fibre 3501 at junction point 3510. The electric signal is not propagated into non-conductive fibre 3501 at junction point 3510 because non-conductive fibre 3501 cannot conduct electricity. Junction point 3510 can refer to any point where adjacent conductive fibres and non-conductive fibres are contacting each other (e.g. touching). In the embodiment shown in FIG. 35A, non-conductive fibre 3501 and conductive fibre 3502 are shown as being interlaced by being knitted together. Knitting is only one exemplary embodiment of interlacing adjacent conductive and non-conductive fibres.

It should be noted that non-conductive fibres forming non-conductive network 3506 can also be interlaced (e.g. by knitting, etc.). Non-conductive network 3506 can comprise non-conductive fibres (e.g. 3501) and conductive fibres (e.g. 3514) where the conductive fibre 3514 is electrically connected to conductive fibres transmitting the electric signal (e.g. 3502).

In the embodiment shown in FIG. 35A, the electric signal continues to be transmitted from junction point 3510 along conductive fibre 3502 until it reaches connection point 3511. Here, the electric signal propagates laterally (e.g. transverse) from conductive fibre 3502 into conductive fibre 3509 because conductive fibre 3509 can conduct electricity. Connection point 3511 can refer to any point where adjacent conductive fibres (e.g. 3502 and 3509) are contacting each other (e.g. touching). In the embodiment shown in FIG. 35A, conductive fibre 3502 and conductive fibre 3509 are shown as being interlaced by being knitted together. Again, knitting is only one exemplary embodiment of interlacing adjacent conductive fibres.

The electric signal continues to be transmitted from connection point 3511 along the electric pathway to connector 3504. At least one fibre of network 3505 is attached to connector 3504 to transmit the electric signal from the electric pathway (e.g. network 3505) to connector 3504. Connector 3504 is connected to a power source (not shown) to complete the electric circuit.

FIG. 35B shows an exemplary woven configuration of a network of electrically conductive fibres 3555. In this embodiment, an electric signal (e.g. current) is transmitted to conductive fibre 3552 from a power source (not shown) through a first connector 3555, as controlled by a controller 3558. The electric signal is transmitted along the electric pathway along conductive fibre 3552 past non-conductive fibre 3551 at junction point 3560. The electric signal is not propagated into non-conductive fibre 3551 at junction point 3560 because non-conductive fibre 3551 cannot conduct electricity. Junction point 3560 can refer to any point where adjacent conductive fibres and non-conductive fibres are contacting each other (e.g. touching). In the embodiment shown in FIG. 35B, non-conductive fibre 3551 and conductive fibre 3502 are shown as being interlaced by being woven together. Weaving is only one exemplary embodiment of interlacing adjacent conductive and non-conductive fibres.

It should be noted that non-conductive fibres forming non-conductive network 3556 are also interlaced (e.g. by weaving, etc.). Non-conductive network 3556 can comprise non-conductive fibres (e.g. 3551 and 3564) and can also comprise conductive fibres that are not electrically connected to conductive fibres transmitting the electric signal.

The electric signal continues to be transmitted from junction point 3560 along conductive fibre 3502 until it reaches connection point 3561. Here, the electric signal propagates laterally (e.g. transverse) from conductive fibre 3552 into conductive fibre 3559 because conductive fibre 3559 can conduct electricity. Connection point 3561 can refer to any point where adjacent conductive fibres (e.g. 3552 and 3559) are contacting each other (e.g. touching). In the embodiment shown in FIG. 35B, conductive fibre 3552 and conductive fibre 3559 are shown as being interlaced by being woven together. Again, weaving is only one exemplary embodiment of interlacing adjacent conductive fibres.

The electric signal continues to be transmitted from connection point 3561 along the electric pathway through a plurality of connection points 3561 to connector 3554. At least one conductive fibre of network 3555 is attached to connector 3554 to transmit the electric signal from the electric pathway (e.g. network 3555) to connector 3554. Connector 3554 is connected to a power source (not shown) to complete the electric circuit.

In accordance with an embodiment, there is provided a method of forming an electric heating (warming) textile based product (e.g. a garment or article) having an integrated heating circuit pattern (e.g. electric pathway) to any one of a first and a second broad surface of a fabric body (of a textile-based product). The integrated heating circuit pattern (e.g. electric pathway) is configured to produce localized heating of the fabric body upon application of electrical current to the circuit pattern. Using an interconnected courses and Wales in a knit structure the integrated conductive layer is configured to allow the formation of the circuit pattern (e.g. electric pathway) that is robust, flat pliable heating (warming) element that can be manufactured and readily integrated to a textile product (fabric based product) to form a fabric article. The flexible nature of the conductive layer provides good dexterity when the heating (warming) element is used in any textile article such as jacket, a glove or other article of clothing in which flexibility is useful. The conductive knit layer formed in the seamless knit structured layer can also be readily configured in various circuit patterns and geometries, e.g., to provide differential heating to different areas of an article, as will be discussed further below.

As such, one or more of the segments can be embodied as a heating segment and/or and an EMS/TENS/ENS segment, based on the construction of the fibres making up the segment as well as the amount and/or duration of power applied to the segment. It is recognized that for a pair of segments in the conductive pathway, one of the segments can be used to transfer power to the other segment being use as the heating segment and/or EMS/TENS/ENS segment. In this manner, the power is applied to selected areas of the garment as either 1) a segment configured as a conductive bus or pathway for simply transferring power to adjacent segments in the electric pathway made up of the segments or 2) a segment configured as a heating element and/or EMS/TENS/ENS element. As such, in order to selectively apply power to selected areas of the textile product in order to provide heat and/or electrical stimulation to the user's body adjacent to those selected areas, the electrical resistance of the segment configured as a conductive bus or pathway would be less that the resistance of the segment configured as a heating element and/or EMS/TENS/ENS element. It is also recognized that in terms of electrical stimulation, the electrical resistance of the segment configured as a conductive bus or pathway would be different from the electrical resistance of the segment configured as the EMS/TENS/ENS element, in order to facilitate selective application of the desired electrical stimulation only to those areas of the textile product containing the segment(s) configured as the EMS/TENS/ENS element. It is also recognized that the segment configured as a conductive bus or pathway could be composed of insulated conductive fibres (in order to inhibit application of electrical stimulation to the skin of the user adjacent to the segment configured as a conductive bus or pathway) while the segment configured as the EMS/TENS/ENS element would include uninsulated conductive fibres (in order to facilitate application of electrical stimulation to the skin of the user adjacent to the segment configured as the EMS/TENS/ENS element).

The conductive fibres of the layer includes metalized textile yarns, metal yarns, filaments selected from the group consisting of (or including) metalized textile yarns, metalized plastic materials, metals and metal foils (in any combination and/or permutation), and any equivalent thereof. These fibres can also be insulated or uninsulated as desired.

The method further includes forming an article of clothing including the seamless fabric body. The forming step (e.g. integration) includes shaping the integrated circuit pattern (e.g. electric pathway) to conform to the shape of the seamless knit article of clothing. The article of clothing includes an article selected from the group consisting of (or including) gloves, socks, sweaters, jackets, shirts, pants, hats, and footwear, etc., and any equivalent thereof

By varying the effective electricity-conducting volume, e.g., the cross-sectional area, of the heating (warming) element in selected regions, the level of heat generation (e.g. resistance) can be controlled. The effectiveness and amount of heat generated in this integrated heating circuit (e.g. electric pathway) in the textile article can be adjusted by adjustment of variation of the width and/or length of the conductive structure. For example, in a heating (warming) element for use in a shoe, the volume of the heating (warming) element in the region of the toes can preferably be less than its volume in the heel region, thus creating greater resistivity in the region of the toes and greater heat generation. Similarly, for use in gloves, the effective volume of the heating (warming) element in the region of the fingers can preferably be less (for greater resistivity and heat generation) than in the palm region.

By varying the effective electricity-conducting volume, e.g., the cross-sectional area, of the EMS/TENS/ENS element in selected regions, the level of electrical stimulation generation (e.g. applied shock) can be controlled. The effectiveness and amount of electrical stimulation generated in this integrated circuit (e.g. electric pathway) in the textile article can be adjusted by adjustment of variation of the width and/or length of the conductive structure configured as the EMS/TENS/ENS element. For example, in a EMS/TENS/ENS element for use in a shoe, the volume of the EMS/TENS/ENS element in the region of the toes can different than the volume of the other segments (e.g. conductive bus element) in the heel region, thus creating greater electrical stimulation in the region of the toes. Similarly, for use in gloves, the effective volume of the EMS/TENS/ENS element in the region of the fingers can preferably different than for other segments (e.g. conductive bus element) in the palm region, thus providing for greater electrical stimulation applied in the region of the fingers over that of the other segments in the palm region. It is also recognized that that conductive fibres of the other segments (e.g. conductive bus element) can be insulated to inhibit application of the electrical stimulation to the adjacent skin of the user of the textile product.

The method can further include configuring the integrated circuit pattern in seamless garments or textile article to include areas of relatively higher resistivity and areas of relatively lower resistivity to provide predetermined regions of relatively higher and relatively lower localized heating (also useful in varying the level of electrical stimulation when certain segments are configured as EMS/TENS/ENS elements). The predetermined areas of relatively higher and relatively lower resistivity are provided by varying the cross-sectional area (another option is the density of the knit/weave pattern of the segment, another option is the amount of conductive verses non-conductive fibres present in the segment) of one or more selected regions of the circuit pattern. The predetermined areas of relatively higher and relatively lower resistivity are provided by varying the conductivity (via cross sectional area, knit density, number of conductive fibres present in the segment, etc.) of one or more selected regions of the conductive layer.

The method can further include configuring the circuit pattern to place the areas of relatively higher resistivity adjacent a wearer's extremities or closer to skin or tailored for specific location on the body when the article of clothing is worn, and/or to place the areas of relatively higher resistivity adjacent regions of the wearer's body where blood flow is close to the skin surface when the article of clothing is worn.

In another embodiment, the hole could be mesh or translucent fabric that provides sufficient optical transparency for the functioning of the optical sensor.

In another embodiment, the connector could be magnetic, other type of physical connector and can be made out of varying conductive materials. In another embodiment, the connector could be analogous in structure to a stereo jack, meaning that two separate electrical connections, e.g. both negative and positive, can be provided by one connector.

In another aspect, it is understood that the distribution network can be used to send signals to multiple connection points, e.g. TENS or EMS signals. In another aspect, it is understood that the distribution network can be used to sense signals from the multiple connection points. In another aspect, it is understood that the fabric or garment connection points can be mixed with conductive fabric sensors and/or electrodes. In another aspect, it is understood that separate networks electrically isolated networks can exist on the garment or fabric at the same time. In one embodiment, there can be a power distribution network and an electrode network. In another aspect, a grid like pattern of conductive yarns can be provided in the first and third layers of fabric. This would allow the connection of connectors at any point where there is connection to the desired electrically conductive yarns of the specific layer

The weight of the garment is measured in GSM (gram square meter). Density can be measured (denier), measuring unit for thickness thread (grams per 0 meters of lineal length).

A factor associated with the existing technology is that (A) the many thicker conductive yarns do not work with some types of garment manufacturing machines (such as, the SANTORINI™ machines), (B) the yarns can physically feel too rough to wearer of garment. An acceptable or usable yarn can include silver-coated nylon thread for heating of the garment. In accordance with aspects, (B) changing shape or knit surface area of heating elements, (B) thinner areas are for heating as they have higher resistance (e.g. about 7 ohms), (C) wider areas are for transmitting electricity as a bus because they are lower resistance (e.g. about 2 ohms), (C) can be used to balance electrical load among different heating channels, and control where heat is generated. Balancing of load is also applicable for the EMS/TENS/ENS elements present in the electrical/conductive pathway comprising a plurality of differently configured segments of differing resistivity.

A factor associated with the existing technology includes stretching fabric that can change resistance (of the fabric): (A) usually when the fabric is stretched, the resistance can change; (B) change density of knit (size of loop affects density, light loop—high density, loose loops—lower density, can affect resistance).

A factor associated with the existing technology is electrical balancing to solve heat generation: (A) calculating resistance to balance out the electrical load using battery and electronic circuit to control heat and temperature; (B) balancing the load to control where the heating is generated; (C) attempt to account for stretching of fabric and change in resistance; (D) weave is changing and that can affect resistance; (E) prior art deals with a single heat control (low/med/high).

A factor associated with the existing technology is how the wearer of the garment is affected by the heat being generated: (A) if you overheat the heart, the body thinks it's hot and the extremities don't get heated up; (B) want to heat the body in zones, extremities vs core chest (e.g. elderly/worker outside, e.g. overcome the “chilling effect”). The solution is to solve (A) with specific zones and regions for targeted heating, or differing levels of heat generation; (B) less heat in the core, more heat at the extremities; (C) with a single power source and control system; (D) adjust heating power; (E) previous problems: multiple leads/multiple heat elements (cumbersome/expensive); (F) feature: multiple heating zones at graduated temperature based on differential heating or heating; (G) feature: responsive heat that incorporates body heat or responsive heat that heats extremities vs just the core

A factor associated with the existing technology is short circuit heat generation: (A) excessive sweating can result in shorting the circuit, and harming the wearer; (B) prior art: insulated yarn can damage insulation; (C) use electrical circuit methods to detect shorts; (D) can be mitigated using knitting techniques FIG. 8 of insulating non-conductive yarn, and then run the conductive thread through the eyes of the FIG. 8; (E) use wicking threads to wick moisture and reduce moisture in garment

A sensor (e.g. one or more segments of the conductive pathway) with various weaknesses is configured to move differently than the fabric attached to the sensor. A solution provides: (A) yarn for wicking; (B) about 0.01 ohms; (C) dense kitting to maintain position; (D) maintain a constant resistance due to the manner in which the sensor deforms and the knit is designed.

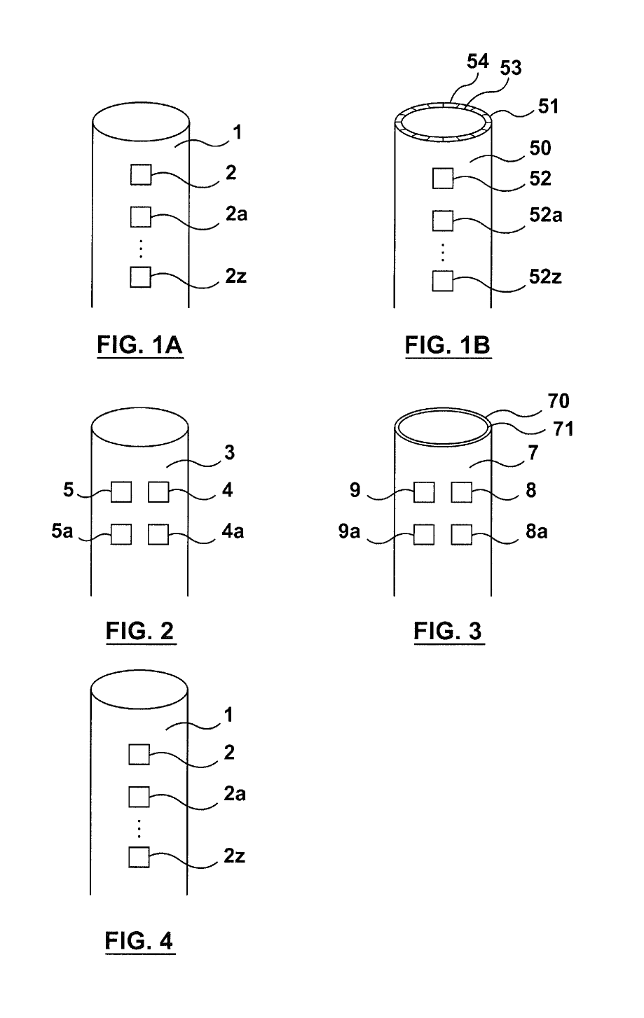

FIGS. 1A and 1B depict views of embodiment of a textile-based product (such as, a knitted garment fabric). With reference to the embodiment as depicted in FIG. 1, there is depicted a seamless sleeve 1 knitted or woven or combination with integrated conductive electrodes g. conductive material 2, 2A . . . 2Z as segments) in a desired pattern or as required for stimulation and/or signal to be conveyed to the user. The desired pattern is aligned along a longitudinal direction.

FIG. 2 depicts a view of an embodiment of a textile-based product (such as, a knitted garment fabric). With reference to the embodiment as depicted in FIG. 2, there is depicted a seamless sleeve 3 knitted or woven or combination with integrated conductive electrodes conductive material 4, 4A, 5, 5A as segments) in a desired pattern and/or distribution or as required for stimulation and/or signal to be conveyed to the user. The pattern extends along a longitudinal direction.

FIG. 3 depicts a view of an embodiment of a textile-based product (such as, a knitted garment fabric). With reference to the embodiment as depicted in FIG. 3, there is depicted a seamless sleeve 7 knitted or woven or combination with integrated conductive electrodes e.g. conductive material 8, 8A, 9, 9A as segments) in a desired pattern or as required for stimulation and/or signal to be conveyed to the user. The desired patterns are in longitudinal direction as well as a horizontal direction. An insulator yarn (that is, a non-electrically conductive yarn) is positioned on the outer layer 70 and part of the inner layer 71 in between the conductive section. This is done in such a way that the pattern of the conductive section can be made in a plaited knit (a circular knit, warp knit or a seamless knit, etc.) where the conductive yarn is positioned in the inner side of the plaited knit construction layer (e.g. in the case where the fabric layer contains multiples of fibres constructed using the interlacing technique (e.g. knitting, weaving) for the network of fibres.

FIG. 4 depicts a view of an embodiment of a textile-based product (such as, a knitted garment fabric). With reference to the embodiment as depicted in FIG. 4, there is provided a seamless sleeve 5 knitted or woven or combination with integrated conductive electrodes (e.g. conductive material 6, 6A . . . 6Z as segments) in a pattern and/or distribution or as required for electronic stimulation and/or a signal to be conveyed to the user. The desired patterns are aligned along a longitudinal direction as well as a horizontal direction. Such a construction (configuration) can have an insulator yarn (that is, a non-electrically conductive yarn) positioned on the outer side chasing the ambient environment, and is configured to reduce risk of electrical short.

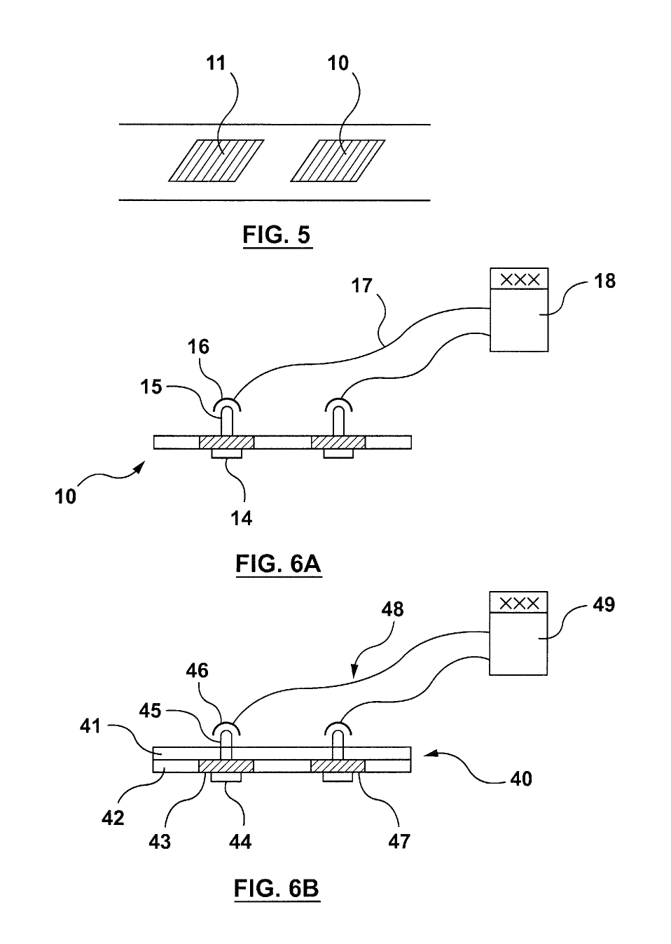

FIG. 5 depicts a view of an embodiment of a textile-based product (such as, a knitted garment fabric). With reference to the embodiment as depicted in FIG. 5, there is provided a seamless sleeve knitted or woven or combination with integrated conductive electrodes 10, 11 (as segments) in a pattern or as required for electronic stimulation and/or for a signal to be conveyed to the user. The pattern is along either a longitudinal direction and/or a horizontal direction. This is done in such a way that the pattern of the conductive section is made in a plaited knit (a circular knit, a warp knit or a seamless knit) where the conductive yarn is positioned in the inner side of the plaited knit construction.

FIG. 6A depicts a view of an embodiment of a textile-based product (such as, a knitted garment fabric). With reference to the embodiment as depicted in FIG. 6A, a seamless sleeve knitted or woven or combination with integrated conductive electrodes (as segments) positioned in a desired pre-determined pattern or as required. The integrated (e.g. knit or woven as comprising/part of the layer) conductive electrodes are configured for use with (to be operatively connectable to) a stimulation signal and/or a signal to be conveyed to the user. The desired pattern is aligned along either along a longitudinal direction and/or a horizontal direction. This is done in such a way that the pattern of the conductive section can be made in a plaited knit (a circular knit or a warp knit or a seamless knit) where the conductive yarn is in the inner side of the plaited knit construction. In case of a single jersey knit or a single layer of wrap knit where the conductive segment is exposed to the body (the skin of the user) as well as the ambient environment. Preferably, insulation is provided by gluing (attaching) a non-conductive layer to the outer side of the conductive segment.

The connection of the conductor segment (the electrical conduit to the power supply) to the electrode segment (that is, the square mat of conductive material facing the skin of the wearer or user) can include any type of conductive physical mechanism, such as a snap connector (quick snap connector), a conductive snap connector with a female portion having an insulator facing the skin of the user (as depicted in FIG. 6A as item 14, and/or as depicted in FIG. 6B as item 44), a conductive wire, a conductive adhesive material, a conductive paste, a sewing portion, a stitching, and any equivalent thereof. For integrated or interlaced fibres, the conductor segment and the electrode/heating segment are knit or woven as part of the fabric layer and as such make up the conductive pathway of having segments of varying resistance to facilitate application of the power transmitted to through the conductive pathway to selected segments (e.g. electrode/heating segment) as heat/electrical stimulation adjacent to specified portions of the user's body.

The connector can be connected directly (or indirectly) to the electrode or to a conductive knitted yarn(s) (as a knitted course(s) integrated with the electrode that can be made during the knitting process). In accordance with an embodiment, the heating circuit can be connected either in series or parallel (or any combination thereof). The resistant yarn (wire) can be non-insulated (preferred option) in a parallel circuit, an insulated resistant yarn (wire) in a series circuit (preferred), and any equivalent thereof.

The electrical heating/stimulation circuit can be knit as integral part of the sleeve or any type of garment or apparel, can be attached (affixed, coupled) to the garment, and any equivalent thereof.

The electrode(s) (i.e. electrical stimulation segments) of the EMS device can be knitted (or woven, etc. or otherwise integrated/interlaced) at a different location of the electrical heating/stimulation circuit. Both electrodes of the EMS device can be positioned above the heating circuit or on both sides of the heating circuit (such as, north and south to the heating element, and not above the planar heating circuit).

The sections related to the connection to the EMS device can be described as following: the connection of the conductor segment (the conduit to the power supply) to the electrode segment (the square mat or patch of conductive material facing the skin of the user) can include any conductive physical mechanism, such as a snap, a snap connector, a conductive snap with a female connector portion having an insulator facing the skin of the user (as depicted in FIG. 6A as item 14 and/or as depicted in FIG. 6B as item 44), a conductive wire, a conductive adhesive material, a conductive paste, a sewing, a stitching, a combination of mechanical device and/or chemical device, and any equivalent thereof

The connector can be attached directly (or indirectly) to the electrode segment, can be attached to a conductive knitted yam(s) (as a knitted course(s) integrated with the electrode during the knitting process), and any equivalent thereof.

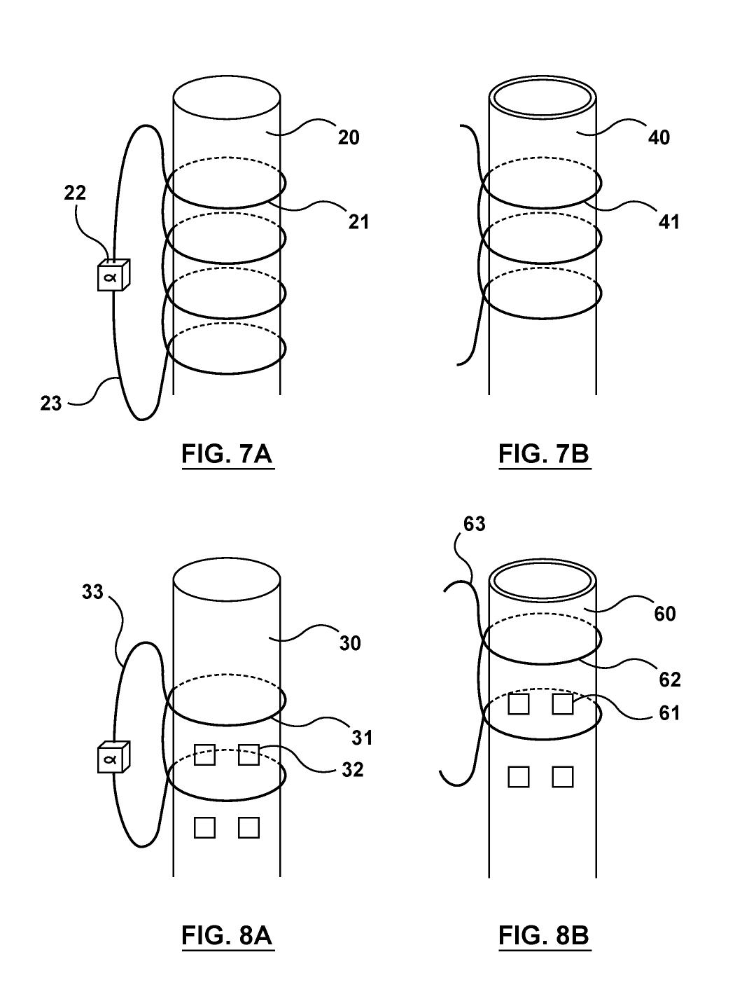

FIG. 7A depicts a view of an embodiment of a textile-based product (such as, a knitted garment fabric). With reference to the embodiment as depicted in FIG. 7A, an integrated heating system is integrated in (one) a seamless silhouette garment. The silhouette garment is a garment having outline, outline shape of the user. The silhouette garment can be constructed in conjunction with electrical stimulation electrodes segments. Adding an electrical heating system into a sleeve, brace or pad can provide further enhanced healing of an aching muscle (of the user). The electrical conducting yarn and/or wire have a predetermined electrical resistance that is configured to generate heat upon connecting the electrical conducting yarn to a power supply. The power supply includes a lithium ion battery having an operating range from about 3.6 Volts DC to about 14 volts DC. The electrical resistance wire can be made of (can include) a multifilament stainless steel arrangement, fine copper wires and/or silver plaited nylon, or any other conductive yarns having a resistance and/or an impedance from between about 0.1 ohms per lineal meter to about 0 ohms per lineal meter, or of any predetermined lineal resistance.

FIG. 7A depicts a view of an embodiment of a textile-based product (such as, a knitted garment fabric). With reference to the embodiment as depicted in FIG. 7A, an integrated heating system is integrated in a seamless silhouette (a garment having outline, outline shape of the user), which is constructed with electrical stimulation electrodes. The addition of an electrical heating system into a sleeve, brace or pad can enhance further the healing of aching muscle. The electrical conducting yarn and/or wire of the heating segment(s) has a predetermined electrical resistance that is configured to generate heat upon connecting the electrical conducting yarn (knitted fabric) to a power supply. The power supply can operate better with a using a lithium ion battery (having a range of about 3.6 Volts to about 14 Volts). The electrical resistance wire can include (can be made of) a multifilament stainless steel, fine copper wires, a silver plaited nylon, or any other conductive yarns having resistance and/or an impedance between about 0.1 ohm per lineal meter to about 0 ohms per lineal meter or of any predetermined lineal resistance. The textile material having the electrical resistance wire embedded therein can be as single knit (such as, a single jersey) or a plaited knit, etc.

FIG. 8A depicts a view of an embodiment of a textile-based product (such as, a knitted garment fabric). With reference to the embodiment as depicted in FIG. 8A, an arrangement is provided for healing aching muscle or an inflamed joint (of the user). The arrangement includes integrating (embedding) a muscle stimulation system and/or an electrical heating as selected heating/stimulation segments of the complete conductive pathway in the same textile unit (knitted fabric garment) as shown in a symmetrically organized separation and/or pattern.

FIG. 8B depicts a view of an embodiment of the knitted garment fabric. With reference to the embodiment as depicted in FIG. 8B, an arrangement is depicted for further enhancement of healing aching muscle and/or inflamed joint (of the user). The arrangement includes integrating (embedding) a muscle stimulation system and/or an electrical heating as selected heating/stimulation segments of the complete conductive pathway in the same textile unit (knitted fiber portion) as shown in an asymmetrically organized separation or pattern.

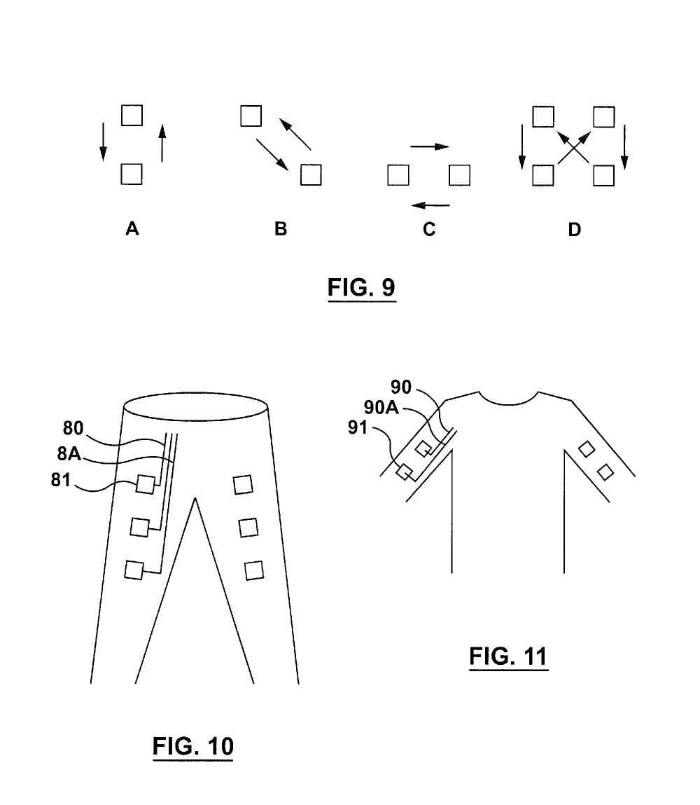

FIG. 9 depicts a view of an embodiment of a textile-based product (such as, a knitted garment fabric). With reference to the embodiment as depicted in FIG. 9, there is provided an integrated seamless structure having an electrically conductive segment positioned on (in or at) the inner layer of a spacer fabric or a sleeve.

FIG. 10 depicts a view of an embodiment of a textile-based product (such as, a knitted garment fabric). With reference to the embodiment as depicted in FIG. 10, an application of EMS with or without heating system is provided with the knitted garment fabric. The knitted garment fabric layer is manufactured by a knitting process. The knitted garment fabric includes a knitted web, such as tights, seamless stockings, and yoga pants, a compression sock, a seamless tubular structure, etc. The electrical pathway includes an electrically conductive knitted portion (segment) configured to be electrically conductive. The electrical pathway is configured to lead to a central power supply and a controller via selected bus/conductor segments of the complete conductive pathway (i.e. of different resistance or otherwise using insulated conductive fibres to those fibres of the heating/EMS/ENS/TENS segment(s)). The controller can be attached (directly or indirectly) to the power supply. A wireless system can activate and/or control the controller (if so desired).

FIG. 11 depicts a view of an embodiment of a textile-based product (such as, a knitted garment fabric). With reference to the embodiment as depicted in FIG. 11, the knitted garment fabric is used with an EMS or a TENS device either with or without a heating system. The knitted garment fabric has a knitted material (formed by a knitting process). The knitted garment fabric is configured to form a T-shirt (or an exercise shirt, a sports bra, a seamless tubular structure worn for the torso. The electrical pathway is knitted with conductive segments (electrodes as well as conductor/bus segments having different resistivities in order to selectively apply the power transmitted to selected adjacent areas of the user's skin). The electrical pathway having knitted conductive segments lead to (are configured to attach to) a power supply and a controller. The controller can be attached (directly or indirectly) to the power supply. A wireless system can activate and/or control the controller (if so desired).

Referring to the embodiments as depicted in FIG. 1A and FIG. 1B, the knitted garment fabric includes a stretchable sleeve 1 (a knitted stretchable sleeve). The stretchable sleeve 1, 50 can be called a knit. Preferably, the stretchable sleeve contains the SPANDEX™ material, at any predetermined SPANDEX™ count and/or at any predetermined stretch-recovery property. For instance, the stretchable sleeve 1, 50 can include a single jersey knit, a plaited jersey or a spacer fabric and any equivalent thereof. Preferably, the stretchable sleeve includes an inter-connecting yarn as a pillar (as depicted as 51 in FIG. 1B) with inner layer 53 and outer layer 54. In accordance with an option, the stretchable sleeve includes a circular knit (also called a warp knit), a seamless circular knit, or warp knit, etc., containing the SPANDEX™ material for body forming and/or full body impression.

The knitted garment fabric (such as, the stretchable sleeve) is constructed of (include any one of) (A) a non-electrically conductive textile yarn (such as, a synthetic fiber polyester material, a nylon material, a polypropylene material and any equivalent thereof) (B) a natural fiber (such as, cotton, wool, silk and any equivalent thereof), and/or (C) a regenerated cellulosic material (such as, rayon and any equivalent thereof) and/or any combination and permutation of the (A), (B) and (C).

Referring to the embodiment as depicted in FIG. 1A, the stretchable sleeve contains (includes) a section of an electrically-conductive material (reference is made to 2, and item 2A and FIG. 1A). The electrically-conductive material is integrally knitted with the stretchable sleeve (during the knitting process for manufacturing the knitted garment section of the knitted garment fabric. The stretchable sleeve (also called a knitted garment section) includes a circular knit, a warp knit, a seamless knit, and any equivalent thereof).

Referring to the embodiment as depicted in FIG. 1A and FIG. 2, the electrically-conductive material can form any predetermined shape (such as a round shape, a square shape, a rectangular shape) and at any predetermined distribution (orientation), such as (A) extending along a longitudinal direction (depicted as 2, 2A to 2Z in FIG. 1A) or (B) extending along a horizontal pattern (depicted as 4 and 5 in FIG. 2). The predetermined surface area of the electrically-conductive material can be formed in a range of about 0.2 inches by about 0.2 inches to about 6.0 inches by about 6.0 inches (approximately).

A similar predetermined pattern of the conductive section of the knitted garment fabric can be made in a plaited knit (a circular knit, a warp knit or a seamless knit) in which the conductive yarn is positioned (A) in the inner side of the plaited knit construction (as shown in FIG. 3 and FIG. 5), (B) at any predetermined pattern longitude (depicted as item 6, item 6A to item 6Z in FIG. 4), (C) other pattern (depicted as item 8, item 8A, item 9, item 9A in FIG. 3), and any equivalent thereof. An insulator yarn (a non-electrical conductive yarn) is positioned on the outer layer (depicted as item 71 in FIG. 3) and is part of the inner layer in between the conductive section (depicted as item 70 in FIG. 3).

Having the insulator yarn (the non-electrically conductive yarn) positioned on the outer side chasing the ambient environment, to reduce risk of electrical short (depicted as item 41 over the layer 42 or the layer 41 over the conductive segment 43 in FIG. 4). For the case where the knitted garment fabric includes a single jersey knit or single layer of wrap knit, the conductive segment is exposed to the body as well as the ambient environment (depicted as item 13 in FIG. 6A). It can be preferred to provide insulation by adhering a non-conductive layer to the outer side of the conductive segment (depicted as item 13 in FIG. 6A).

Referring to the embodiment as depicted in FIG. 1B, the conductive segment is positioned on the inner layer (depicted as item 53 in FIG. 1B) of a spacer fabric, or a sleeve (depicted as item 50 in FIG. 1B). The conductive yarn and/or wire can be made of (can include) a multifilament conductive wire having stainless steel or copper (and any equivalent thereof). The conductive yarn can be made of synthetic yarn and/or fiber coated with the conductive material. The conductive material can include silver, copper, graphene, polyaniline, polypyrrole, and any equivalent thereof. Polypyrrole (PPy) is a type of organic polymer formed by polymerization of pyrrole. The conductive material can be (A) embedded in the fiber during the extrusion process throughout (at least in part) the whole cross-section of the fiber and/or (B) on the outer layer in the core sheath. Optionally, another conductive yarn is made of (includes) a synthetic fiber (such as, nylon, polyester, and any equivalent thereof) in which a conductive material can include copper (such as, the CUPRON™ yarn, and any equivalent thereof) and/or silver (such as, the X-Static™ yarn, and any equivalent thereof) where the conductive material is deposited and reacts with the surface of the fiber. The conductive segment is depicted as item 13 in FIG. 6A, and as item 43 in FIG. 6B.

The conductive segment is connected through a physical attachment such as, a snap connector (depicted as item 14 in FIG. 6A, and as item 44 in FIG. 6B). The conductive textile material (depicted as item 43 in FIG. 6B, and as item 13 in FIG. 6A) can include the electrode segment. The snap connector protrudes through the textile. The snap connector has a non-electrical conductive sleeve or a fabric (depicted as item 15 in FIG. 6A, and as item 45 in FIG. 6B). The snap connector is connected (depicted as item 16 in FIG. 6A, and as item 46 in FIG. 6B) to a power supply or a controller (depicted as item 18 in FIG. 6A, and as item 45 in FIG. 6B).

In accordance with an embodiment, the sleeve is positioned over the aching muscle or the joint (of the user), or a similar layer as part of a back brace. The muscle is triggered by nerve impulse to contract in response to electrical stimulation. The electrical stimulation is controlled by the controller configured to send signals with a variety of frequencies and magnitude thereby stimulating a greater portion of the muscle. The electronic stimulation of the nerve provides analgesic effect to the user.

The modulated electronic stimulation can be in sequence of several options (variable intensity cycling, relatively lower frequency (about one pulse per second) and/or a pulse made of about four seconds of sustained pulses followed by about one second OFF (that is, deactivated), or any other pattern combination including a single frequency and/or a voltage wave form over the whole (entire) treatment session. Preferably, the electrode directly touches the skin (of the user) through the snap connector and/or the conductive textile section as a component of the sleeve, a brace, a pad, and any equivalent thereof

Having variety and sequence of stimulation for longer period can overcome the gradual diminution in response to ongoing stimulus (of electronic signals).

Preferably, the device (depicted as item 18 in FIG. 6A, and depicted as item 49 in FIG. 6B) is powered by a battery (such as, two AAA 1.5 Volt DC alkaline batteries). The output voltage can range from about +Volts DC to about −Volts DC. The frequency range can range from about 1.0 Hertz to about Hertz. The treatment length can be as desired or required (such as, 10 minutes, 30 minutes or 60 minutes). The duration of individual pulses can be in range of about 30 milliseconds to about milliseconds, and can vary from about three Hertz to about 0 Hertz.

In accordance with an embodiment, an electrical heating system is added (incorporated) into the knitted garment fabric (such as, a sleeve, a brace, a pad, and any equivalent thereof). The heating system is configured to enhance further healing effect (therapy) for the aching muscle (of the user). The electrical heating system includes, for instance, an electrical conducting yarn (wire) (depicted as item 21 in FIG. 7A, and as item 41 in FIG. 7B) having a predetermined electrical resistance as the heating element (i.e. heating segment) configured to generate heat in response to connection of the heating assembly to a power supply. The power supply can include a DC battery (such as, a lithium ion battery operating in the range from about 3.6 volts to about 7.2 volts. The battery is depicted as item 22 in FIG. 7A. The electrical heating system can include an electrical resistance wire made of multifilament stainless steel having (for example) about 70 ohms per lineal meter, or any predetermined lineal resistance value, etc.

Referring to the embodiment as depicted in FIG. 7B or FIG. 9, the textile material of the knitted garment fabric (where the electrical resistance wire is embedded therein) includes a single knit (such as, a single jersey), a plaited knit (depicted as item 40 in FIG. 7B), a spacer fabric as depicted in FIG. 9, and any equivalent thereof

In accordance with an embodiment, the electrical resistance wire includes a knitted material (such as, a circular knit, a wrap knit, a seamless knit, and any equivalent thereof). For instance, the electrical wire can include a non-insulated material or an insulated material (such as, PVC material or other suitable material) covering the conductive material.

Referring to the embodiment as depicted in FIG. 8A and FIG. 8B, a further enhancement for healing the aching muscle or the inflamed joint (of the user) can be accomplished by embedding the electronic simulation system (for muscle stimulation) and/or the electrical heating in the same textile unit (that is, in the knitted garment fabric).

Referring to the embodiment as depicted in FIG. 9, there muscle stimulation directions are depicted in relation to the aching muscle of the user. The shape can include any one of a longitudinal shape (depicted as item A in FIG. 9), a diagonally extending shape (as depicted as item B in FIG. 9), a horizontally extending shape (depicted as item C FIG. 9) and/or a complex shape (depicted as item D in FIG. 9), and any combination and/or permutation thereof.

In accordance with an embodiment, the knitted garment fabric includes a textile material having an antimicrobial property. In addition, the knitted garment fabric includes a textile material configured to manage water (such as, removing sweat away from the skin of the user to keep the skin relatively dry).

The medical treatment device (such as the electronic simulation device, either with or without a heating system) can be incorporated in the knitted garment fabric. The knitted garment fabric includes a knitted material (manufactured by a knitting process). The knitted garment fabric includes a shirt (as depicted in FIG. 11), a tight (as depicted in FIG. 10) a compression sock. The conductive segment (depicted as item 10, depicted as item 11 in FIG. 5) which can be the electrode or in combination of snap, upon connecting to a power supply with a controller.

In accordance with another embodiment, the electrical pathway (depicted as item 80 in FIG. 8B and FIG. 10) is knitted with the conductive segments (electrodes, conductors/bus or differing resistivities or otherwise differing electrical simulation potential). The electrical pathway can be leading to a power supply and/or a controller. The controller can be attached to the power supply. A wireless system can activate and control the controller.

The knitted garment fabric, the knitted electrical circuit (e.g. electric pathway) and the integrated knitted heating system can be knitted (formed on a seamless knitting machine or assembled through a cut and sew process), where the SPANDEX™ material can be incorporated in the knit structure to keep the electrodes and the electrical pathway in close proximity to the skin in the predetermined location. The knitted electrical pathway can be made of bare conductive wire, insulated conductive wire, partially insulated (metered insulation) and any equivalent thereof

The knitted garment fabric can be used for electrical stimulation for therapy and/or pain relief, and can be used in conjunction with monitoring sensors to provide haptic feedback. For instance, a soldier (who has been inactive while on guard duty) can receive a light electrical stimulation (from the knitted garment fabric) to keep the soldier attentive. A patient sitting and/or lying in one position without movement is prone to bed sores and/or ulcers, and a smaller electrical signal can stimulate the patient to move. The inactivity of the user (wearer) can be easily monitored through sensors in the controller module. The knitted garment fabric can be used on a patient with Alzheimer's or any other form of cognitive deterioration.

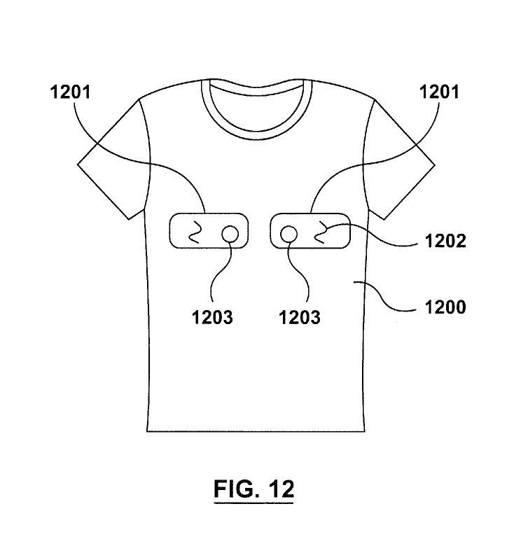

FIG. 12 depicts a view of an embodiment of a textile-based product (such as, a knitted garment fabric).

Referring to the embodiment as depicted in FIG. 12, the segemt(s) of the conductive knit portion 1201 of garment 1200 is used as both a sensor and an electrode. The conductive knit portion 1200 comprises conductive stitching 1202, and connectors 1203 and is used as an electrode for any one of electrical muscle stimulation (EMS) and/or transcutaneous electrical nerve stimulation (TENS). It is understood that to effectively deliver signals for an EMS device and/or a TENS device, the placement of the electrodes can be important relative to the part of the wearer's body. EMS provides involuntary muscle stimulation. The electrodes are place to activate the muscle. TENS stimulates nerves in order to relieve pain. Specific placement of the fabric electrode on the body can be required. In an embodiment, the garment is structured to ensure that the fabric electrode maintains a desired position on the wearer's body within a spatial tolerance, as the body moves and the garment (compression garment) deforms in response to the body movement. For instance, the using the LYCRA™ material for tighter fit (on the body) in order to create more friction to assist the fabric electrode stays in contact with the body at the desired location. In another embodiment, the conductive fabric patch is used as both a sensor and as an electrode as required (e.g. to sense body signals or information about the body, and then to provide stimulation in response to those signals.

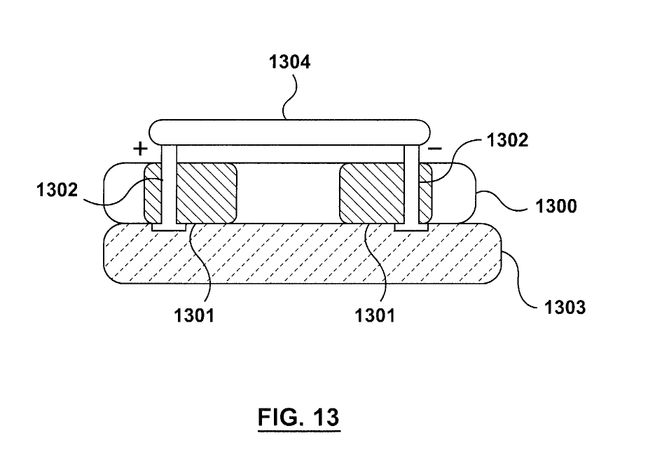

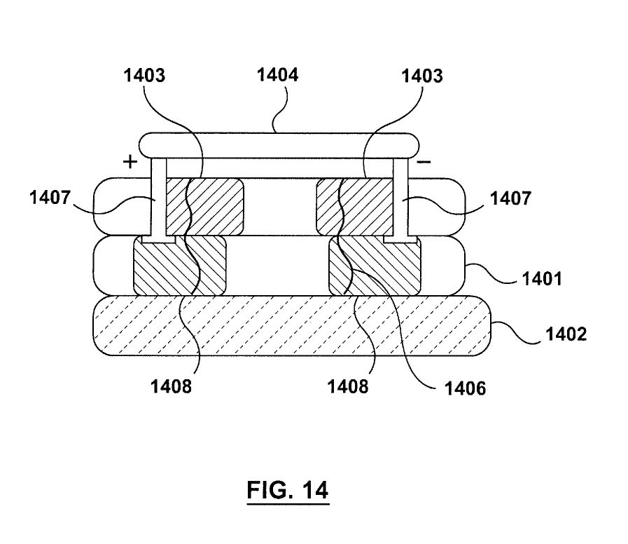

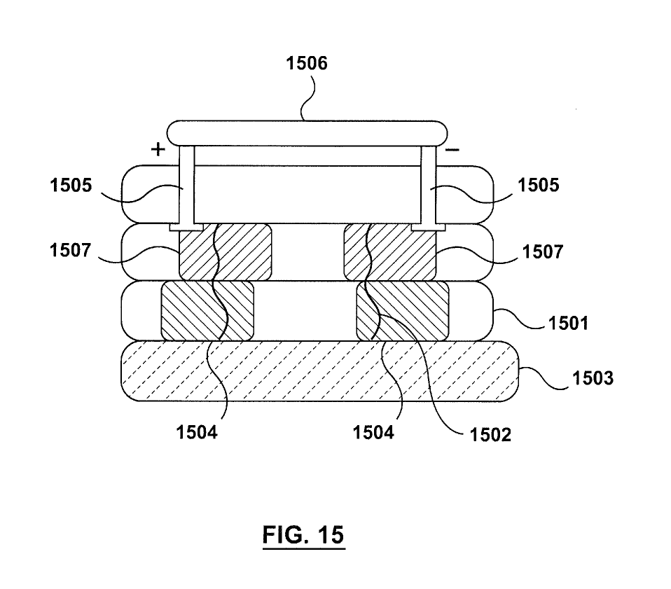

FIG. 13, FIG. 14 and FIG. 15 depict views of embodiments of a textile-based product (such as, a knitted garment fabric).

Referring to the embodiments as depicted in FIG. 13, FIG. 14 and FIG. 15, these embodiments provide for relatively precise placement of sensors on the knitted garment fabric relative to the body of the wearer once the knitted garment fabric is worn (just so).

Referring to the embodiments as depicted in FIG. 14 and FIG. 15, improved placement of the sensor during movement of the wearer is provided because the electronics is located on the outer layer of the knitted garment fabric.

Referring to the embodiment as depicted in FIG. 13, the knitted garment fabric includes a one layer knitted fabric portion 1300. The one layer (single layer) includes a conductive fabric area (i.e. segment) configured to sense and/or function as an electrode 1301 (to deliver EMS or TENS electrical stimulation). The connectors 1302 include metal snaps. The metal snap is configured to make electrical contact with the knitted fabric portion. The metal snap can make contact the skin of the wearer 1303 to enhance conductivity (there can be some frictional discomfort to the wearer). The layer surface for touching the skin (of the user) includes fabric or knitted properties or construction that allows the fabric conductive patch to maintain spatial position within a tolerance at a desired point on the body (of the wearer). Also included is controller 1304.

Referring to the embodiment as depicted in FIG. 14, the knitted garment fabric includes a two layer knitted fabric incorporating a multiple conductive fabric areas. The layer of fabric 1401 in contact with the skin 1402 contains a knitted fabric conductive patch 1406, with no metal contact, for increased comfort of the wearer. The metal snap is connected to the electronic controller 1404 for providing EMS or TENS stimulation. The metal snap (electrical connector) is electrically and physically connected to the second layer of fabric at the conductive fabric patch. Then, the two conductive fabric patches 1403 make electrical contact (either by friction or can be enhanced by sewing with conductive thread). The first layer closet to the skin 1401 (of the user) has fabric or knitted properties or construction that allows the fabric conductive patch to maintain spatial position within a tolerance at a desired point on the body (of the user). Connectors 1407 and sensor electrodes 1408 are also shown. As such, each of the layers of the textile product comprise a network of fibres interlaced to one another (e.g. knitted, woven), such that each of the network of fibres contains a separate conductive pathway having a plurality of electrically interconnected segments of varying/differing resistivity, in order to selectively apply the power transmitted through the conductive pathway to those segments configured as heating/EMS/TENS/ENS elements (also referred to as electrodes) while using the other segments (e.g. electrical conductors/connectors/bus) to only transfer the power from the power source to the heating/EMS/TENS/ENS elements. As such, the other segments (e.g. electrical conductors/connectors/bus) are configured via their resistivity to be used only for transfer of power and as such are not configured for transmission of the power of a desired/configured level/amplitude (as either heat or electrical stimulation) to the adjacent skin of the user of the textile product.

Referring to the embodiment as depicted in FIG. 15, the knitted garment fabric includes a three layer knitted fabric incorporating multiple conductive fabric areas. The first layer closet to the skin (of the user) 1501 has fabric or knitted properties or construction that allows the fabric conductive patch 1502 to maintain spatial position within a tolerance at a desired point on the body 1503. The three layers cooperate to allow the electrode 1504 to be located at a specific location and for the attachment of the electronics to be located at another location. The middle layer 1507 provides electrical connection between the physical connector 1505 to the electronics 1506 located on the third or outside layer and/or the first layer next to the skin. In this manner, it is recognized that there can be, for multiple interlaced/integrated layers of the textile product, an intervening layer between a particular conductive pathway of one layer and the user's skin. It is recognized that the intervening layer can also have a conductive pathway separate from the conductive pathway in the one layer.

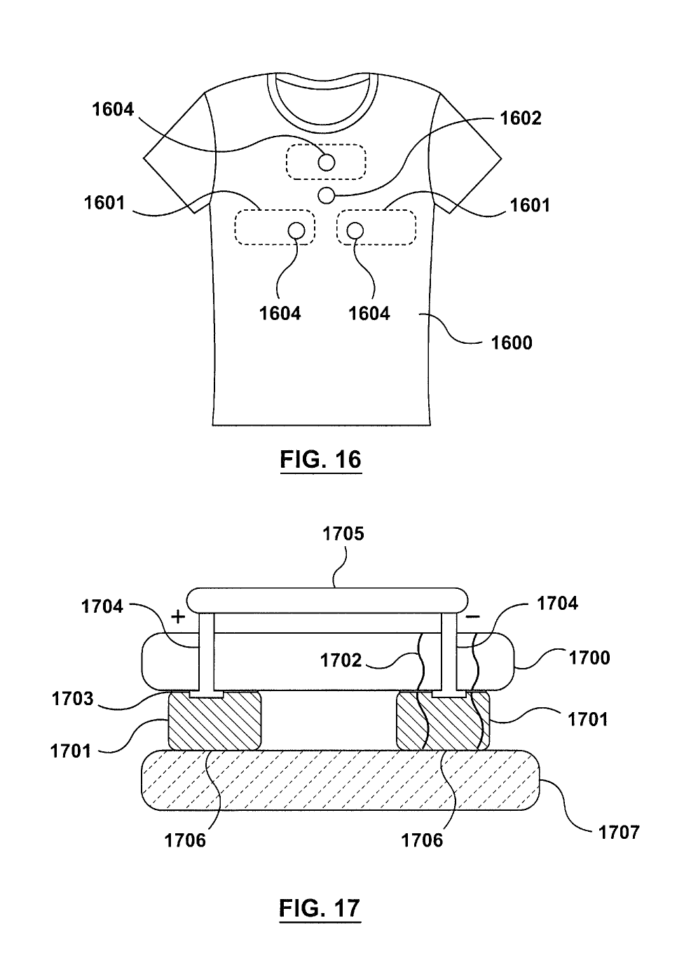

FIG. 16 depicts a view of an embodiment of a textile-based product (such as, a knitted garment fabric). Referring to the embodiment as depicted in FIG. 16, the knitted garment fabric 1600 includes three fabric conductive patches 1601 operating as sensors and/or fabric electrodes. A hole 1602 is formed in the garment. The hole 1602 is configured to cooperate with an optical sensor or to provide an electrode direct contact with the skin. The fabric conductive patch is not integral or knit into the one layer of fabric. The fabric sensor is knit separately or provided separately and then is attached to the garment (through a cut and sew operation). The fabric conductive patch is connected to the fabric of the garment by a stitch or through an adhesive. For the case where electrical conductivity is required, a conductive yarn/thread or conductive adhesive is used. Connectors 1604 are also shown. As such, this embodiment is not considered as having conductor/bus segments and heating/EMS/ENS/TENS segments integrated/interlaced (e.g. knitted, woven) into the fabric layer of the textile product, which is contrary to the textile product and described fabric layer of FIGS. 35A, 35B and 24, 25.

FIG. 17 depicts a view of an embodiment of a textile-based product (such as, a knitted garment fabric). Referring to the embodiment as depicted in FIG. 17, the knitted garment fabric 1700 includes a fabric conductive patch 1701 that is not integral or knit into the one layer of fabric. The fabric sensor is knit separately or provided separately, and then is attached to the garment (through a cut and sew operation). The fabric conductive patch 1701 is connected to the fabric of the garment by a stitch 1702 or through an adhesive 1703. For the case where electrical conductivity is required, a conductive yarn/thread or conductive adhesive is used. Connectors 1704, controller 1705, and electrodes 1706 are also shown. The wearer is shown as 1707. As such, this embodiment is not considered as having conductor/bus segments and heating/EMS/ENS/TENS segments integrated/interlaced (e.g. knitted, woven) into the fabric layer of the textile product, which is contrary to the textile product and described fabric layer of FIGS. 35A, 35B and 24, 25.

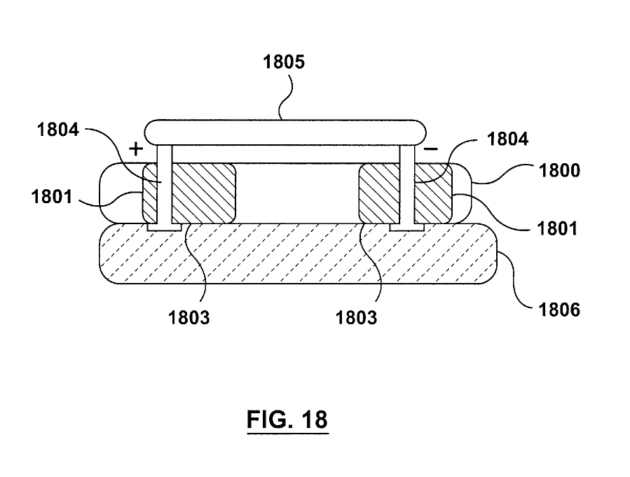

FIG. 18 depicts a view of an embodiment of a textile-based product (such as, a knitted garment fabric). Referring to the embodiment as depicted in FIG. 18, the knitted garment fabric 1800 includes the fabric conductive patches 1801 that are knitted or woven directly in to the fabric 1800. It can be preferable to have the fabric conductive knit or woven directly into the fabric of the garment for efficiency and cost-effectiveness reasons (for example, by increasing automation and decreasing manual cut and sew operations). For the one layer, two layer or three layer embodiments, the sensor or electrodes 1803 can be incorporated. Connectors 1804 and controller 1805 are also shown. The wearer 1806 is also shown. As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

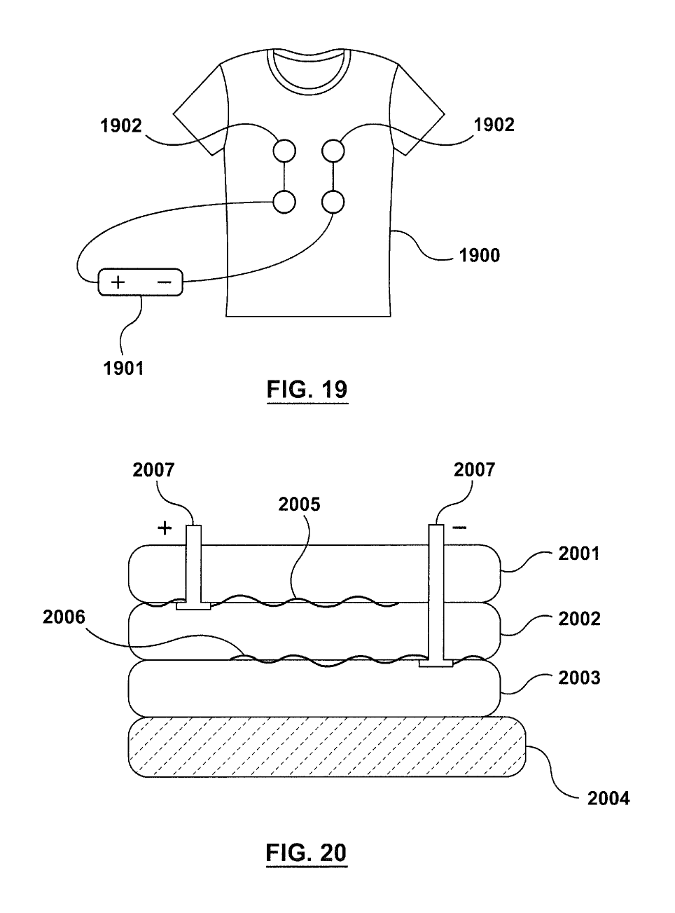

FIG. 19 depicts a view of an embodiment of a textile-based product (such as, a knitted garment fabric). Referring to the embodiment as depicted in FIG. 19, the knitted garment fabric 1900 includes a power distribution circuit. A battery or source of electricity 1901 is provided. In use, the battery 1901 provides electrical current via the electrical connectors 1902 (terminals) mounted to the garment 1900 and to the electrical distribution circuit. The connector can include a knit fabric patch.

FIG. 20 depicts a view of an embodiment of a textile-based product (such as, a knitted garment fabric). Referring to the embodiment as depicted in FIG. 20, the knitted garment fabric includes the power distribution garment. There is an outer layer of fabric 2001, a middle layer of fabric 2002, and an inner layer of fabric 2003 next to the wearer's skin 2004. The middle layer of fabric 2002 is configured to provide an electrical insulator. The conductive yarn 2005 is knitted or woven into the outer layer of fabric 2001 on the inside layer facing the middle layer. Another conductive yarn 2006 is knitted on the layer of the inner fabric on the layer facing the middle layer. The two conductive pathways form the positive or negative electrical pathways of the electrical distribution circuit (e.g. electric pathway). The middle layer 2002 provides insulation so that there are no shorts (electrical short circuit) with the two conductive yams or with their respective attached connectors 2007. As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

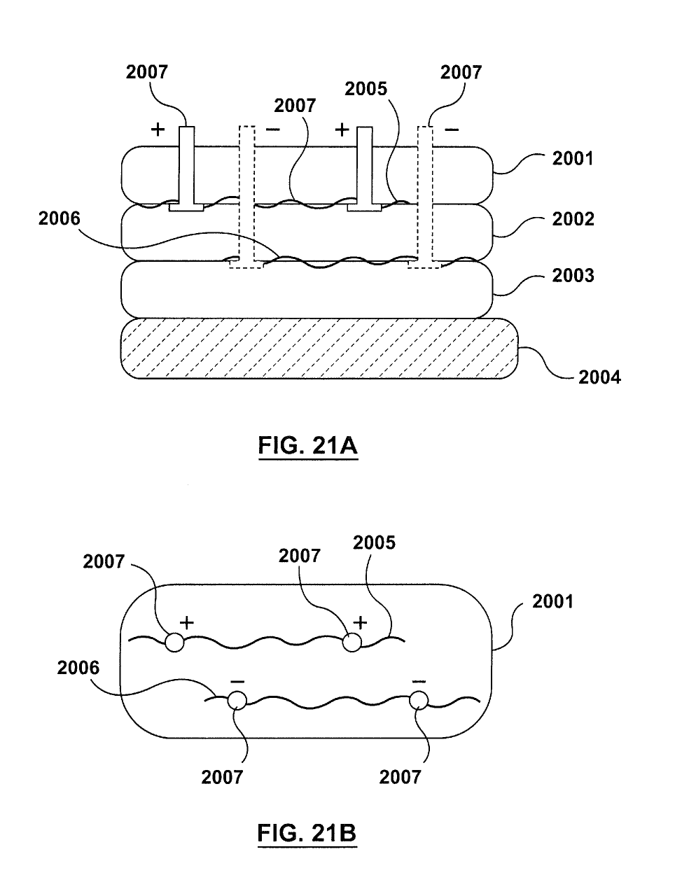

FIG. 21A depicts a view of an embodiment of a textile-based product (such as, a knitted garment fabric). Referring to the embodiment as depicted in FIG. 21A (in a cross-sectional view), the knitted garment fabric includes multiple connectors 2007 provided on the garment. The conductive yams 2005, 2006 run in series or in parallel depending on the desired electrical circuit configuration to each of the connectors. The specific pattern of how the conductive yarns is knit into the garment should inhibit creating shorts (electrical short circuits). There is an electrical insulated effect provided by the middle layer of the garment. The regions of connectors pass from the outside of the garment through the first two layers of the garment. As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

FIG. 21B depicts a view of an embodiment of a textile-based product (such as, a knitted garment fabric). The embodiment as depicted in FIG. 21B represents a corresponding top-view to FIG. 21A).

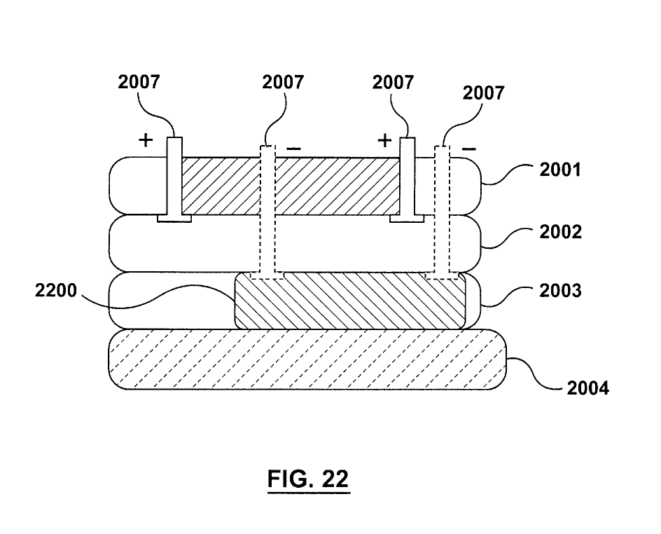

FIG. 22 depicts a view of an embodiment of a textile-based product (such as, a knitted garment fabric). Referring to the embodiment as depicted in FIG. 22, the knitted garment fabric includes a conductive yarn 2200 that is knit into the fabric layer to form an electrical or conductive pathway.

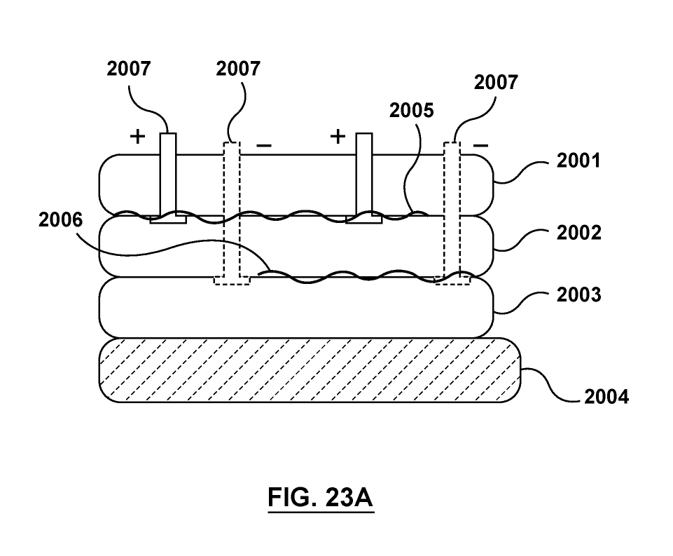

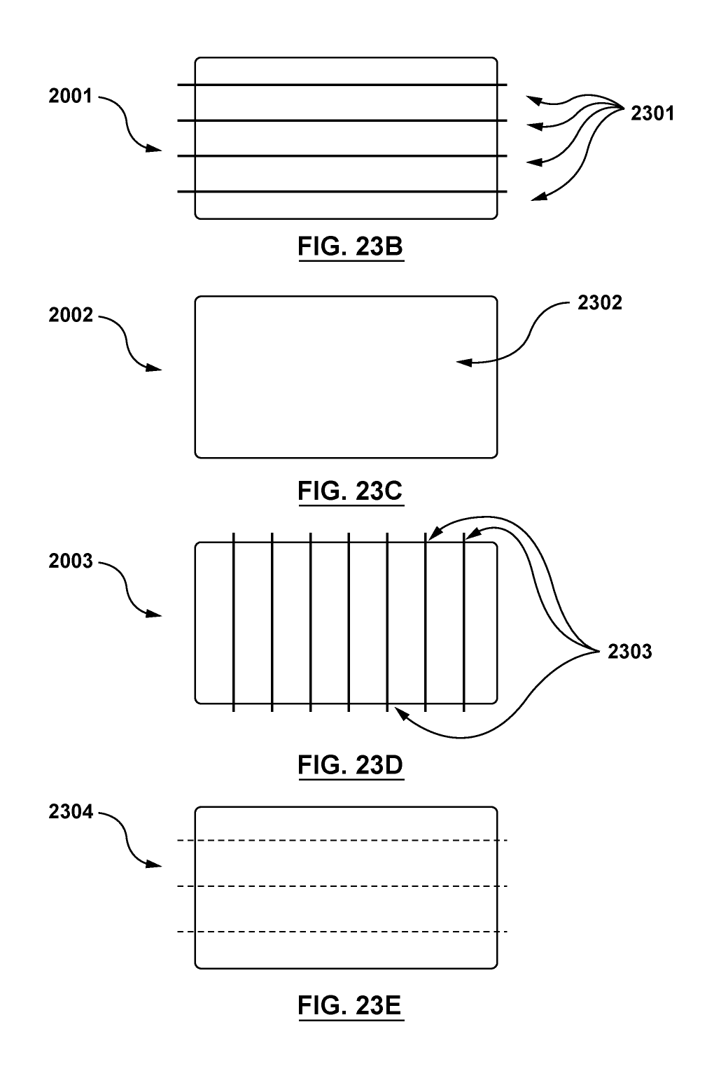

FIG. 23A depicts a view of an embodiment of a textile-based product (such as, a knitted garment fabric). Referring to the embodiment as depicted in FIG. 23A, the knitted garment fabric includes a middle layer that includes a dielectric material. A fabric is knitted or woven to provide a dielectric effect. This enables the fabrication of knit or woven fabric capacitor.

FIGS. 23B to 23E depict views of embodiments of a textile-based product (such as, a knitted garment fabric). There is depicted a capacitive layer. The first layer includes a grid of lines representing conductive yarns 2301 (horizontal); the second layer includes a dielectric layer 2302; and the third layer includes a grid of lines representing conductive yarns 2303 (vertical), A capacitive fabric is shown as 2304. As such, this embodiment is considered as having conductor/bus segments and heating/EMS/ENS/TENS segments integrated/interlaced (e.g. knitted, woven) into the fabric layers of the textile product, which is similar to the textile product and described fabric layer of FIGS. 35A, 35B and 24, 25.

FIG. 24 depicts a view of an embodiment of a textile-based product (such as, a knitted garment fabric). Referring to the embodiment as depicted in FIG. 24, the knitted garment fabric includes options for heating/electrical stimulation (as described above). Various patterns of knit or woven yarn of resistive yarns are depicted. The shapes or the knit or woven pattern affect the resistance in that area and allow for the control, within a tolerance, of the heating effect generated by the resistance yarns. For example, the thinner sections have a higher resistance are generate more heat. The wider sections have a lower resistance and generate less heat. The medium width or surface area sections generate a medium amount of heat. This allows a fully automatic knit or woven method for providing and controlling where heat is provided in a garment. A skilled person would understand that corresponding electrical power source and control circuitry would be required. A problem solved is that no wire has to be soldered or attached, and that heat control in multiple regions of the garment could be provided by adjusting the overall resistivity of each branch or network. Considerations can be provided for the parallel or serial electrical characteristics of each branch (is desired). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 25.

FIG. 25 depicts a view of an embodiment of a textile-based product (such as, a knitted garment fabric). Referring to the embodiment as depicted in FIG. 25, the knitted garment fabric includes the heating/electrical stimulation options as described above. Various patterns for producing heat/stimulation and modifying resistivity can be provided. As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24.

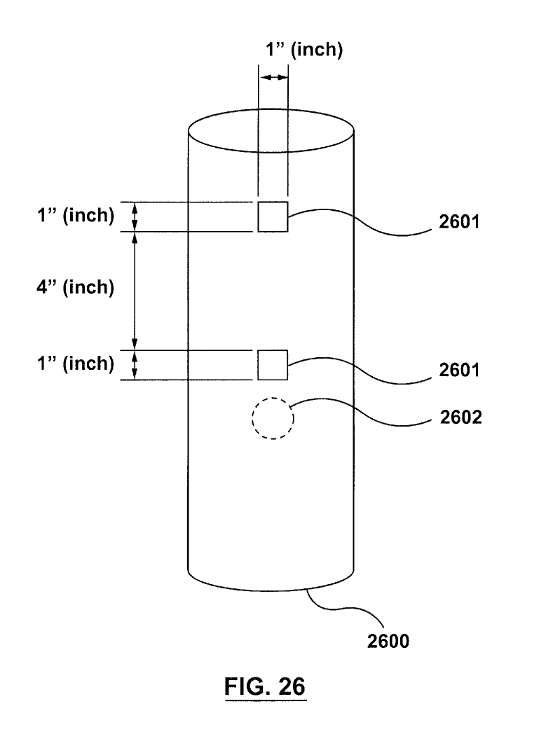

FIG. 26 depicts a view of an embodiment of a textile-based product (such as, a knitted garment fabric). Referring to the embodiment as depicted in FIG. 26, the knitted garment fabric includes a knee brace 2600. An embodiment of a compression garment is shown. This compression garment is adapted for use on a knee (e.g. knee patella 2602), and there are fabric conductive patches 2601 shown. These fabric conductive segments can be used for providing EMS or TENS signals. A skilled person would understand that the compression garment can be adapted for other body parts.

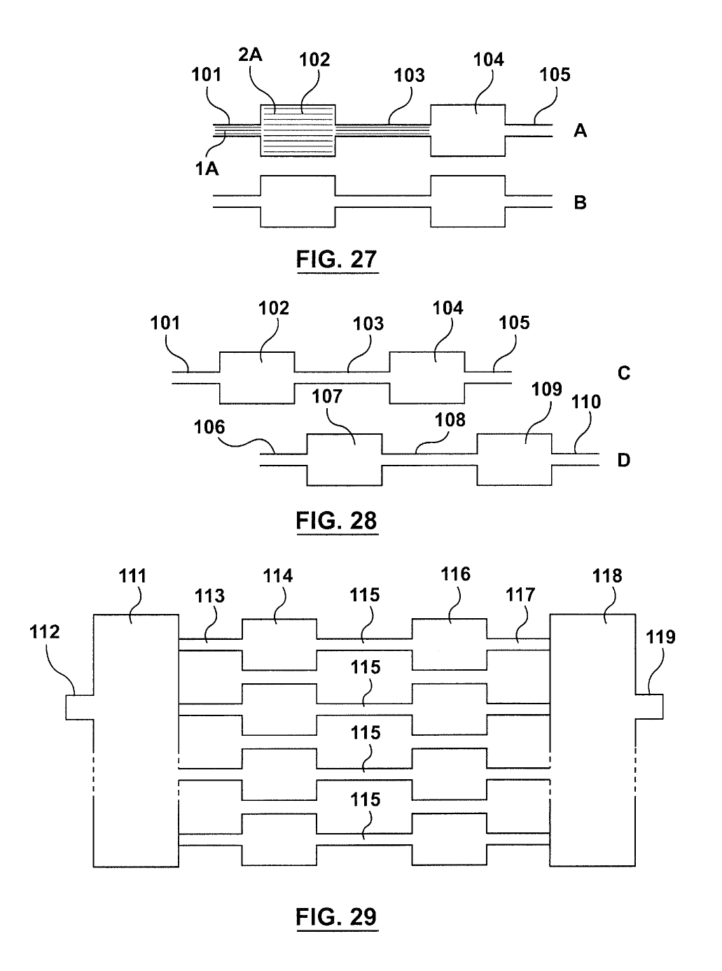

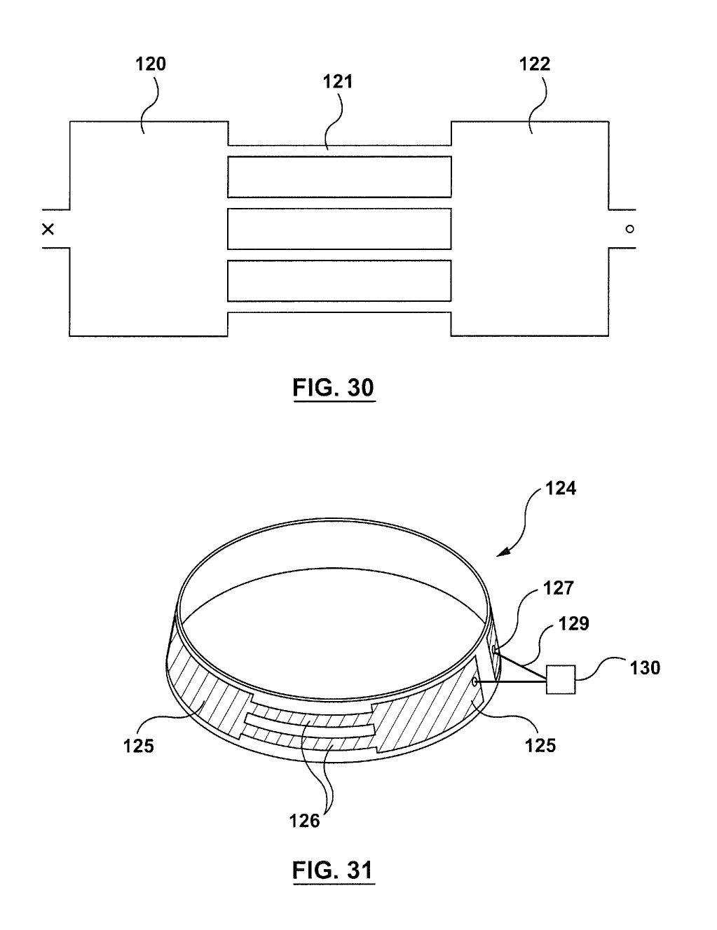

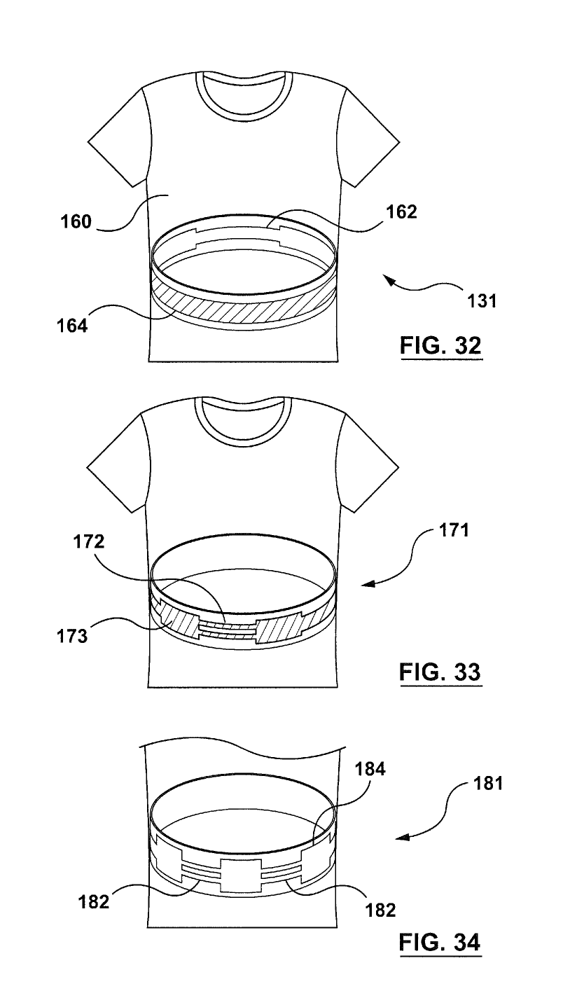

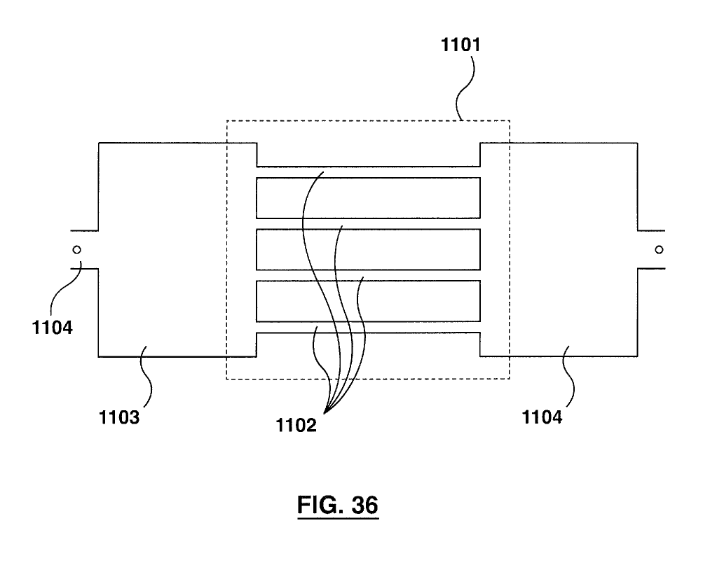

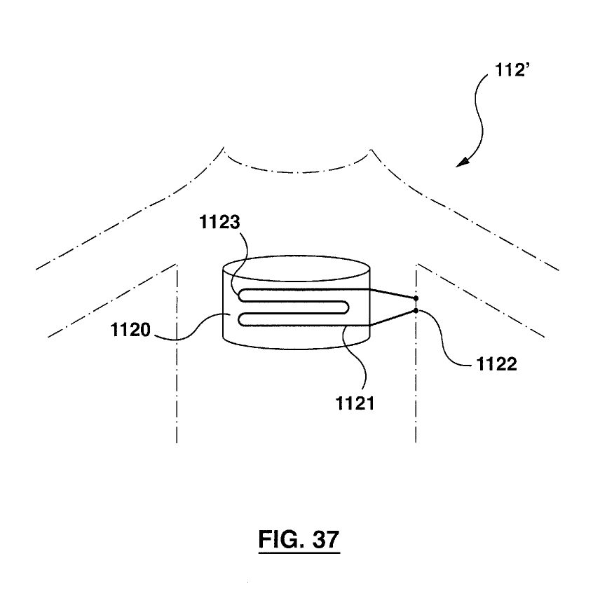

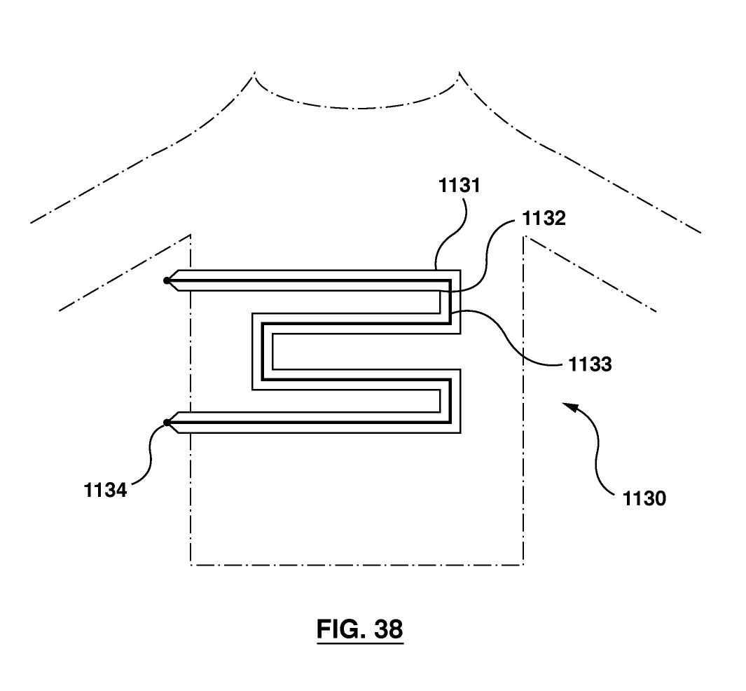

FIG. 27, FIG. 28 and FIG. 29 depict views of embodiments of a textile-based product (such as, a knitted garment fabric). FIG. 30 and FIG. 31 depict views of embodiments of a textile-based product (such as, a knitted garment fabric). FIG. 32, FIG. 33 and FIG. 34 depict views of embodiments of a textile-based product (such as, a knitted garment fabric). FIGS. 35A and 35B depict views of an embodiment of a textile-based product (such as, a knitted garment fabric). FIG. 36 depicts a view of an embodiment of a textile-based product (such as, a knitted garment fabric). FIG. 37 depicts a view of an embodiment of a textile-based product (such as, a knitted garment fabric). FIG. 38 depicts a view of an embodiment of a textile-based product (such as, a knitted garment fabric).

The electrically heated garment (such as a jacket, etc.) can be powered by a battery. The electrically heated garment can include an electrical resistance panel (e.g. as a pad having the conductor/bus segments and heating/EMS/ENS/TENS segments integrated/interlaced (e.g. knitted, woven) into the fabric layer of the panel, which is similar to the textile product and described fabric layer of FIGS. 35A, 35B and 24, 25. s such the panel is attached to the inner side of the garment (e.g. jacket). As such, the textile product can be embodied as an insert to an existing garment or other textile product. The electrical or resistive panel is connected to a power supply (such as, battery), and is configured to be activated by a controller. This system can be less desirable and has few deficiencies, such as: (A) losing heat to the cold ambient environment; (B) the sensorial thermal (skin sensing) is significantly lower due to the heating element being far away from the skin/body. To overcome these and other deficiencies, the resistive panel is configured such that the resistive panel (in use) consumes more power (electrical power from battery or power source) and/or the resistive panel is operated for a relatively longer heating time (which adversely affects the longevity or reduces the battery usage life).

Another approach to overcome the above deficiency is to attach the heating/stimulation panel to the inner layer (such as, a shirt or underwear). This attachment can be made of incompatible materials and can result in a stiffer hand (feel) which can cause irritation, bruising, chaffing and/or skin irritation, etc., for the user.

In accordance with an embodiment, the preferred electrical heated/stimulated system can be integrated and is an integral part of the first layer, with similar property of the stretch, recovery and comfort level. Having the integrated electrical heating/stimulation panel (circuit, textile circuit) positioned relatively closer to the skin of the user can enhance the thermal sensing as well as reduce the heat loss to the environment (having other fabric/garment layers on top of the first layer entraps the heat and reduces the heat loss to the environment). This arrangement can require less power (lower battery usage, less electrical current is consumed), and accordingly can increase the time of usage of the battery and/or the effective time for which a user can use the electrically heatable garment.

In accordance with an embodiment, the first layer can be made on a seamless knitting machine where the electrical circuit (also called the electrical heated section (e.g. electric pathway)) is an integral part of the seamless garment, with identical or similar physical properties (stretch, recovery, weight, tensile strength, flex, etc.). The seamless knitting machine can include a circular knit machine manufactured by the SANTONI™ Company, a flat-bed knit machine manufactured by the SHIMA SEIKI® Company, the seamless warp knit machine, and other seamless garment machines, and any equivalent thereof.

In accordance with an embodiment, the knit structure can include a single jersey, a plaited jersey, a terry-plaited jersey, and any equivalent thereof. The plaited jersey can contain nylon or polyester on one side with the SPANDEX™ material covered with nylon or polyester (and any equivalent thereof). The covered SPANDEX™ yarn can be on every feed or on any predetermined pattern or repeat.

The nylon or polyester yarn can be of different fineness (denier) ranging from about 10 Denier to about 300 Denier singles or multiple filaments or two-plied or three-plied o r any combination and/or permutation as required (and any equivalent thereof) for the final properties of the garment or textile structure.

Similarly, the SPANDEX™ material can be selected from about 10 Denier to about 200 Denier and can be covered with nylon or polyester having fineness of about 10 Denier to about 200 Denier (mono-filament and/or multifilament yarns), any combination and/or permutation (and any equivalent thereof) as required for the final properties of the garment or textile structure.

Additionally, the knitted seamless shirt, garment, textile, and any equivalent thereof, can be dyed in atmospheric-dyeing machine (at a temperature of about 212 Fahrenheit) before or after heat setting done with dry heat ranging from about 325 Fahrenheit to about 400 Fahrenheit or by steaming.

An alternative filament yarn can be used in the construction of the garment (textile) with the integrated heating circuit (e.g. electric pathway). Other yarns that can be used are cotton, rayon, wool, aramid and others and combination (blend) of one or more (and any equivalent thereof).

The heating circuit (i.e. conductive pathway containing multiple segments of varying resistance) is (preferably) integrated in the textile structure (seamless garment, textile, etc.) can be generated through (manufactured with) the use of conductive yarns. The conductive yarns that can be used can have a denier ranging from about 10 Denier to about 2000 Denier with resistance ranging from about 0.1 ohm per meter to about 1000 ohms per meter. Various conductive yarns available for use in building and integrating the resistive electrical circuit into the textile structure are: the X-STATIC® yarns (single-ply, multiple ply, about 50 Denier to about 200 Denier single ply), MAGLON™ yarns (single-ply, two-ply, three-ply), a stainless steel (a mono filament, multi-filaments where the number of filaments can range from about 14 to about 512, and each filament thickness ranging from about 5 microns to about 100 microns), AARCON™ yarns, and other available yarns (such as, copper, indium yarns etc., and any equivalent thereof. The conductive yarns can be combined or bundled to achieve the desired resistive result for developing the integrated heating structure in the garment.

The conductive material can be used as is (bare) or covered with polymer coatings such that the conductive yarns are covered (preferably, fully) in an insulation layer. The insulation can be imparted to conductive yarns with a coating of PVC or any thermoplastic resin (such as, EVA, polyamide, polyurethanes, etc., and any equivalent thereof

The non-conductive yarns (garment body yarns), which make the remainder (those portions of the garment/textile product that contain non-conductive fibres that are not segments in the conductive pathway) of the textile structure or garment, can be selected from available synthetic fibers and yarns, such as polyester, nylon, polypropylene, etc., and any equivalent thereof), natural fiber and yarns (such as, cotton, wool, etc., and any equivalent thereof), a combination and/or permutation thereof, and each as required for the final properties of the garment or textile structure. The garment body yarns can be wrap or plaited during knitting, wrap in a yarn form (twisted at a number of turns per inch as can be required).

The SANTONI® seamless machine is configured to knit in circular knit (using a desired cylinder size), course after course with capability to generate a plain knit or a pattern knit to enhance the user comfort level of the wearer as well, as adding aesthetic and/or a fashion appearance.

The conductive yarn can be incorporated on the face side or the backside (in a plaited construction) or in a single jersey knit where the conductive yarn can be exposed to both sides of the fabric or the face and back of the fabric. The conductive yarn or the electrical resistive yarn or wire is knitted in any predetermined pattern having heating section and a conductive circuit completion section (a, electrical bus) in such a pattern that there is no heating on the connective or conductive circuit completion or conductive section joining the resistive sections (e.g. segments) of the integrated knitted heating circuit (e.g. electric pathway).

In accordance with an embodiment, the heating section (as depicted in FIG. 27 as item 101, item 103, item 105) is made of conductive yarns which can be selected from various conductive yarns described above (the X-STATIC yarn, the MAGLON yarn, stainless steel, copper, ARACON yarn, indium, etc., and any equivalent thereof) in multiple courses attached or interconnected to each other separated by segments 102, 104. The number of conducting courses in this section and the length of the heating section can determine the resistance of the heating segment (the integrated conductive circuit or heating circuit). The resistance of the heating segment is the total addition in ohms of segment 101, segment 103 and segment 105 (Resistance in series).

In accordance with an embodiment, the resistance of the section A and section B when connected by a bus (as depicted in FIG. 29 as item 111, and item 118) as shown in FIG. 29 results in an electrical circuit, where the resistive sections are connected in a parallel electrical circuit. As such, this embodiment is considered as having conductor/bus segments and heating/EN/IS/ENS/TENS segments integrated/interfaced (e.g. knitted, woven) into the fabric layer of the textile product, which is similar to the textile product and described fabric layer of FIGS. 35A, 35B and 24, 25.

In accordance with an embodiment, FIG. 27 and FIG. 28 depict two parallel circuits where (as depicted in FIG. 27 as item A, and item B) the heating elements are parallel to each other. FIG. 28 shows parallel heating unit (as depicted in FIG. 28 as item C, item 101, item 103, and item 105) are staggered to (depicted in FIG. 28 as item D, item 106, item 107, item 108, item 109 and item 110) which generate different levels of heat (watts per square unit area) at the same resistance and same current (amps). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

In accordance with an embodiment, FIG. 30 depicts another parallel electrical circuit made with conductive yarns in the knitted or seamless textile structure where the heating element are made of multiple courses made of conducive yarn (as depicted in FIG. 30 as item 121) and bus segment (as depicted in FIG. 30 as item 120, and item 121) are made of multiple courses of 100% conductive yarn to have a very low electrical resistance. The heating segment (as depicted in FIG. 30 as item 121) can be in symmetrical separation from each other or asymmetrical (different distance from each other). The multiple courses made of conductive yarn are touching each other or inter-connected to each other (as shown in FIGS. 35A and 35B as item 1′ and item 2′) and maintaining conductivity along the courses and the Wales, generating a planar conductive element. FIG. 29 shows three heating segments electrically connected in series (item 113, item 115, item 117) and separated by items 114, 116 and 118 with eight courses each and total length of about six inches. These three segments are in connected in parallel and are made of the four sections as shown in FIG. 29. As such, this embodiment is considered as having conductor; bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

The heating segment in this case (FIG. 29) is made of eight courses of conductive yarn selected from any available conductive yarns or combination of the available yarns. For the case where the resultant structure has a resistance of about 10 ohms and when the resultant structure is connected to a power supply (preferably to about 7.2 volt DC battery, preferably a lithium ion battery or any other power source), the resultant structure can generate about five watts of heat. The bus segment (as depicted in FIG. 29 as item 111, and item 118) is made of multiple courses of the conductive yarn, such that the bus segment generates very low resistance and does not generate heat on connection to the power sources (as depicted in FIG. 29, item 112, item 119) to a power supply (battery or any other power source). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

The seamless knitted shirt (also called a textile structure, a garment) contains a segment having electrical heating components electrically connected in parallel (as depicted in FIG. 31 as item 126, as depicted in FIG. 33 as item 171, as depicted in FIG. 34 as item 182 and item 183) and large section of two buses (as depicted in FIG. 31 as item 125 and item 125′) connected to the power supply (as depicted in FIG. 31 as item 130). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

The band form or illustration of the heating element can be configured such that the heating element can be located at any pre-determined section of the human body, such as the back or kidney area (as depicted in FIG. 32 as item 162, as depicted in FIG. 33 as item 172). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

The heating band form can be an integral section of a shirt or a stand-alone garment. A band can be used as heating brace for the lower or upper back, the joints or the muscles of the user. The electrical heating section (as depicted in FIG. 36 as item 1102) can be protected by lamination of another textile or material patch (as depicted in FIG. 36, as item 1101) on one side or both sides of the textile knit structure and/or construction. The laminated patch can have a water-resistant material or waterproof properties or any other desired properties (stretch, no stretch, abrasion, insulation etc.). The laminate (as depicted in FIG. 36, as item 1101) can be made of a film (such as, polyurethane, mylar (polyester film or plastic sheet), polyester, polypropylene, etc.) or a woven fabric (limited stretch and/or non-stretchable). This laminate can protect the heating elements from excessive abrasion (wet and dry), friction during the laundry and dyeing stage as well as reducing the friction on the conductive yarn elements in the heating segment (as a result of stretch and recovery of the structure). Another way to protect the conductive yarn from abrasion is covering the conductive yarn during the knitting with a non-conductive yarn. The electrical resistance yarn/wire can be knitted in a terry yarn floating over a determined number of Wales (needles), such as one knit and four floats. The floating of the conductive yarn can be on single jersey of plaited jersey at any predetermined length of float and length of the anchor stitch (such as 1 by 1 or 1 by 4, or any other combination). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

The electrical heated/warming textile fabric can be the whole garment (such as a shirt or legging) for casual sports, healthcare, hunting, hiking, climbing, skiing, and military or any other outdoor or indoor use. The electrical heated /warming textile fabric can be used as a heating band like brace or wrap around or sleeve. The textile fabric can be treated for wicking property and/or soil release and/or anti-microbial finish and/or odor repellent finishes.

In accordance with another embodiment, the garment can include a body fitting, a compression seamless shirt/garment, a textile structure with heating element is incorporated in a pocket (sewn in or made on seamless knitting machine) into which a panel (as depicted in FIG. 37 as item 1120) is inserted in a pocket (as depicted in FIG. 38 as depicted in 1123) of shirt (as depicted in FIG. 37 as item 112′). The heating element (as depicted in FIG. 37 item 1121) can be an electrical insulating conductive yarn/wire made by stitching, sewing, embroidery, laying it and securing it. The ends of the heating element (as depicted in FIG. 37 as item 1122) are connected to a power supply like battery. The pocket can be located at any predetermined location (upper back, lower back etc.). As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

Seamless knitting on knitting machines (such as, the SHIMA SEIKI™ machine) can also be used to generate stretch or body fitting shirt or garment or textile structure where the heating element can be made of insulated yarn or wire. The electrical heating can be knit in any pre-determined pattern which can be electrically connected in series (as depicted in FIG. 38 as item 1131) or in parallel (as depicted in FIG. 38 as item 1131, item 1132, item 1133) connected to terminals (as depicted in FIG. 38 as item 1132) to which a power supply can be connected. As such, this embodiment is considered as having conductor/bus segments and heating/EMS/ENS/TENS segments integrated/interlaced (e.g. knitted, woven) into the fabric layer of the textile product, which is similar to the textile product and described fabric layer of FIGS. 35A, 35B and 24, 25.

To get a specific resistance of the total electrical circuit (series or parallel) the following (can be taken into consideration) parameters of length of the knitted conductive or insulated conductive yarn or wire as well as the linear resistance of the wire or conductive yarn (ohms per meter).

This written description uses examples to disclose the invention, including the best mode, and also to enable any person skilled in the art to make and use the invention. The patentable scope of the invention is defined by the claims, and can include other examples that occur to those skilled in the art. Such other examples are within the scope of the claims if they have structural elements that do not differ from the literal language of the claims, or if they include equivalent structural elements with insubstantial differences from the literal language of the claims.

It can be appreciated that the assemblies and modules described above can be connected with each other as required to perform desired functions and tasks within the scope of persons of skill in the art to make such combinations and permutations without having to describe each and every one in explicit terms. There is no particular assembly or component that can be superior to any of the equivalents available to the person skilled art. There is no particular mode of practicing the disclosed subject matter that is superior to others, so long as the functions can be performed. It is believed that all the crucial aspects of the disclosed subject matter have been provided in this document. It is understood that the scope of the present invention is limited to the scope provided by the independent claim(s), and it is also understood that the scope of the present invention is not limited to: (i) the dependent claims, (ii) the detailed description of the non-limiting embodiments, (iii) the summary, (iv) the abstract, and/or (v) the description provided outside of this document (that is, outside of the instant application as filed, as prosecuted, and/or as granted). It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

As such, one or more of the segments can be embodied as a heating segment and/or and an EMS/TENS/ENS segment, based on the construction of the fibres making up the segment as well as the amount and/or duration of power applied to the segment. It is recognized that for a pair of segments in the conductive pathway, one of the segments can be used to transfer power to the other segment being use as the heating segment and/or EMS/TENS/ENS segment. In this manner, the power is applied to select areas of the garment as either 1) a segment configured as a conductive bus or pathway for simply transferring power to adjacent segments in the electric pathway made up of the segments or 2) a segment configured as a heating element and/or EMS/TENS/ENS element. As such, in order to selectively apply power to selected areas of the textile product in order to provide heat and/or electrical stimulation to the user's body adjacent to those selected areas, the electrical resistance of the segment configured as a conductive bus or pathway would be less that the resistance of the segment configured as a heating element and/or EMS/TENS/ENS element. It is also recognized that in terms of electrical stimulation, the electrical resistance of the segment configured as a conductive bus or pathway would be different from the electrical resistance of the segment configured as the EMS/TENS/ENS element, in order to facilitate selective application of the desired electrical stimulation only to those areas of the textile product containing the segment(s) configured as the EMS/TENS/ENS element. It is also recognized that the segment configured as a conductive bus or pathway could be composed of insulated conductive fibres (in order to inhibit application of electrical stimulation to the skin of the user adjacent to the segment configured as a conductive bus or pathway) while the segment configured as the EMS/TENS/ENS element would include uninsulated conductive fibres (in order to facilitate application of electrical stimulation to the skin of the user adjacent to the segment configured as the EMS/TENS/ENS element).

Further embodiments, the textile product can comprise: a non-conductive section comprising a network of non-conductive fibres; and an electric pathway for conducting or transmitting an electrical signal when connected to a power source via a first connector and a second connector, the electric pathway and the non-conductive section integrated into a common layer of the textile, the electric pathway comprising: a first conductive segment of the electric pathway for coupling with the power source via the first connector, the first conductive segment comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source, the first conductive segment having a first electrical resistance; and a second conductive segment of the electric pathway for coupling with the power supply via the second connector, the second conductive segment comprising a second network of conductive fibres having a plurality of third conductive fibres, at least one third conductive fibre coupled to the second connector along the electric pathway, and a plurality of fourth conductive fibres interlaced with the third conductive fibres extending lateral to the pathway, the second conductive segment having a second electrical resistance differing from the first electrical resistance.

Further, the textile product can have the first conductive segment and the second conductive segment arranged in series such that the electric signal is transmitted from the first network of conductive fibres to the second network of conductive fibres. For example, the second conductive segment can be attached directly to the second connector via the at least one third conductive fibre or the second conductive segment being attached indirectly to the second connector via a third conductive segment coupled to the second conductive segment, the third conductive segment directly attached to the second connector.

Alternatively, the first conductive segment can be attached indirectly to the first connector via a third conductive segment coupled to the first conductive segment, the third conductive segment directly attached to the first connector. As such, there can be an intervening conductive segment (e.g. third segment) between the first conductive segment and the first connector attached to the power source. As such, there can be an intervening conductive segment (e.g. third segment) between the second conductive segment and the second connector attached to the power source. Alternatively, there can be an intervening conductive segment attached between the first and second conductive segments.

What is claimed is:

1. A textile product comprising:
a non-conductive section comprising a network of non-conductive fibres; and
an electric pathway for conducting or transmitting an electrical signal when connected to a power source via a first connector and a second connector, the electric pathway and the non-conductive section integrated into a common layer of the textile, the electric pathway comprising:
a first conductive segment of the electric pathway for coupling with the power source via the first connector, the first conductive segment comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source, the first conductive segment having a first electrical resistance: and
a second conductive segment of the electric pathway for coupling with the power supply via the second connector, the second conductive segment comprising a second network of conductive fibres having a plurality of third conductive fibres, at least one third conductive fibre coupled to the second connector along the electric pathway, and a plurality of fourth conductive fibres interlaced with the third conductive fibres extending lateral to the pathway, the second conductive segment having a second electrical resistance differing from the first electrical resistance.

2. The textile product of claim 1, wherein the first conductive segment and the second conductive segment are arranged in series such that the electric signal is transmitted from the first network of conductive fibres to the second network of conductive fibres, the second conductive segment being attached directly to the second connector via the at least one third conductive fibre or the second conductive segment being attached indirectly to the second connector via a third conductive segment coupled to the second conductive segment, the third conductive segment directly attached to the second connector.

3. The textile product of claim 1, wherein the first conductive segment and the second conductive segment are arranged in parallel.

4. The textile product of claim 1, wherein the first and second electrical resistances are proportional to a density of the first and second networks of conductive fibres.

5. The textile product of claim 1, wherein the first and second electrical resistances are proportional to a length of the pluralities of first, second, third and fourth conductive fibres.

6. The textile product of claim 1, wherein the first and second electrical resistances are proportional to a width of the pluralities of first, second, third and fourth conductive fibres.

7. The textile product of claim 1, wherein the plurality of first conductive fibres is interlaced with the plurality of second conductive fibres by knitting or weaving.

8. The textile product of claim 1, wherein the first conductive segment is attached indirectly to the first connector via a third conductive segment coupled to the first conductive segment, the third conductive segment directly attached to the first connector.

9. The textile product of claim 1, wherein the plurality of second conductive fibres extend laterally from the plurality of first conductive fibres at 90°.

10. The textile product of claim 1, wherein the plurality of fourth conductive fibres extend laterally from the plurality of third conductive fibres at 90°.

11. The textile product of claim 1, wherein the network of non-conductive fibres includes non-conductive fibre material that comprises at least one of: nylon; cotton; spandex; polyester; or silk.

12. The textile product of claim 1, wherein each of the networks of conductive fibres includes conductive fibre material comprising at least one of: stainless steel; silver; aluminum; copper; or gold.

13. The textile product of claim 1, wherein the first conductive segment and the second conductive segment are connected to one another by at least one intervening third conductive segment.

14. The textile product of claim 1, further comprising a second electric pathway for conducting or transmitting a second electrical signal when connected to the power source, the second electric pathway and the non-conductive section integrated into the common layer of the textile; the second electric pathway comprising:
a first stimulating conductive segment for coupling with the power supply via a first stimulating connector, the first stimulating conductive segment comprising a first stimulating network of conductive fibres having a plurality of first stimulating conductive fibres, at least one first stimulating conductive fibre coupled to the first stimulating connector along the second electric pathway, and a plurality of second stimulating conductive fibres interlaced with the first stimulating conductive fibres extending lateral to the second electric pathway to transmit the second electric signal from the power source; and
a second stimulating conductive segment as an electrode and for coupling with the power supply via a second stimulating connector; the second stimulating conductive segment comprising a second stimulating network of conductive fibres having a plurality of third stimulating conductive fibres, at least one third stimulating conductive fibre coupled to the second stimulating connector along the second electric pathway, and a plurality of fourth stimulating conductive fibres interlaced with the third stimulating conductive fibres extending lateral to the second electric pathway;
wherein the electrode is configured to deliver the second electric signal to an adjacent underlying body portion of a wearer of the textile.

15. A textile product comprising:
a first conductive segment for coupling with a power supply via a first connector and a second connector attached to an electric pathway, the first conductive segment of the electric pathway comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source, the first conductive segment having a first electrical resistance; and
a second conductive segment of the electric pathway for coupling with the power supply via the second connector_{;} the second conductive segment having a second network of conductive fibres having a plurality of third conductive fibres, at one third conductive fibre coupled to the second connector along the electric pathway, and a plurality of fourth conductive fibres interlaced with the third conductive fibres extending lateral to the pathway, the second conductive segment having a second electrical resistance differing from the first electrical resistance;
the first and second conductive segments of the electric pathway integrated into a common layer of the textile.

16. A textile product comprising:
a non-conductive section comprising a network of non-conductive fibres; and
an electric pathway for conducting or transmitting an electrical signal when coupled to a power source via a first connector and a second connector attached to the electric pathway, the electric pathway and the non-conductive section integrated into a common layer of the textile; the electric pathway comprising:
a first conductive segment of the electric pathway for coupling with the power supply via the first connector, the first conductive segment comprising a first network of conductive fibres having a plurality of first conductive fibres, at least one first conductive fibre coupled to the first connector along the electric pathway, and a plurality of second conductive fibres interlaced with the first conductive fibres extending lateral to the electric pathway to transmit the electric signal from the power source; and
a second conductive segment configured as an electrode of the electric pathway and for coupling via the second connector, the second conductive segment comprising a second network of conductive fibres having a plurality of third conductive fibres, at least one third conductive fibre coupled the second connector along the electric pathway, and a plurality of fourth conductive fibres interlaced with the third conductive fibres extending lateral to the pathway;
wherein the electrode is configured to deliver h electric signal to an adjacent underlying body portion of a wearer of the textile.

17. The textile product of claim 15, wherein the plurality of first conductive fibres is interlaced with the plurality of second conductive fibres by knitting.

18. The textile product of claim 15, wherein the plurality of first conductive fibres is interlaced with the plurality of second conductive fibres by weaving.

19. The textile product of claim 15, wherein the plurality of second conductive fibres extend laterally from the plurality of first conductive fibres at 90°.

20. The textile product of claim 15, wherein the textile product is a garment or an insert to a garment.

21. A mixed layer textile product comprising:
as a first layer, the non-conductive section and the electric pathway as defined in the textile of claim 1; and
as a second layer, the non-conductive section and the electric pathway as defined in textile of claim 15.

22. The mixed layer textile product of claim 21, further comprising a third layer configured as an electric insulator and positioned between the first layer and the second layer.

23. The mixed layer textile product of claim 21, wherein the electric pathway comprises both conductive and non-conductive fibres in the fibre network.
